(19) United States
(12) Patent Application Publication
(10) Pub. No.: US 2012/0211706 A1
(43) Pub. Date: Aug. 23, 2012

(54) POLYDENTATE LIGAND METAL COMPLEX

(75) Inventors: Yusuke Kuramochi, Tokorozawa-shi (JP); Takeshi Ishiyama, Tsukuba-shi (JP); Hideyuki Higashimura, Tsukuba-shi (JP)

(73) Assignee: SUMITOMO CHEMICAL COMPANY, LIMITED, Chuo-ku, Tokyo (JP)

(21) Appl. No.: 13/504,275

(22) PCT Filed: Oct. 25, 2010
(86) PCT No.: PCT/JP2010/068838

§ 371 Date: Apr. 26, 2012

(30) Foreign Application Priority Data
Oct. 28, 2009 (JP) 2009-248144

(51) Int. Cl. C07F 5/00; H01B 1/12

(52) U.S. Cl. 252/519.2; 534/15

(57) ABSTRACT

The present invention provides a metal complex that has excellent durability, and a device and the like having excellent durability that uses such a metal complex. Specifically, the present invention provides a metal complex comprising (a) a polydentate ligand having denticity of five or more that includes a heteroaromatic ring which contains two or more atoms selected from the group consisting of a nitrogen atom, an oxygen atom, and a sulfur atom, and (b) an ion of a metal selected from the group consisting of cerium, praseodymium, ytterbium, and lutetium; a composition comprising the metal complex and a charge transport material; an organic thin film obtained by using the metal complex or composition; and a device obtained by using the metal complex, composition, or organic thin film.

### TECHNICAL FIELD

The present invention relates to a rare earth metal complex comprising a polydentate ligand.

### BACKGROUND ART

Certain types of rare earth metals can be used as the central atom in metal complexes that act as a light-emitting material used in the light-emitting layer of an organic electroluminescent device (which may be referred to as an organic EL device). For example, it is known that a cerium complex that uses a tetradentate ligand including a benzimidazolyl group can exhibit strong luminescence based on 4f-5d transition, and that such a cerium complex can be useful as a material for an organic EL device (Non-Patent Literature 1).

### PRIOR ART LITERATURE

### Non-Patent Literature

Non-Patent Literature 1: Xiang-Li Zheng, Cheng-Yong Su et al., Angew. Chem. Int. Ed., 46, 7399-7403 (2007)

### SUMMARY OF THE INVENTION

### Problem to be Solved by the Invention

However, the metal complexes that exhibit luminescence based on 4f-5d transition such as the cerium complex described in Non-Patent Literature 1 suffer from the problem of low durability.

Therefore, it is an object of the present invention to provide a metal complex having excellent durability.

### Means for Solving Problem

The present invention is as follows.

[1] A metal complex comprising:
(a) a polydentate ligand having denticity of five or more that includes a heteroaromatic ring which contains two or more atoms selected from the group consisting of a nitrogen atom, an oxygen atom and a sulfur atom; and
(b) an ion of a metal selected from the group consisting of cerium, praseodymium, ytterbium, and lutetium.
[2] The metal complex according to [1], wherein the number of said polydentate ligand comprised in said metal complex is one.
[3] The metal complex according to [1] or [2], wherein said heteroaromatic ring is an imidazole ring or a condensed imidazole ring.
[4] The metal complex according to any of [1] to [3], wherein said polydentate ligand is represented by the following formula (1):

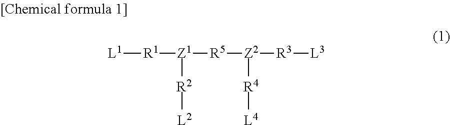

wherein

R¹, R², R³, R⁴, and R⁵ each independently represent a divalent group or a direct bond;

Z¹ and Z² each independently represent a nitrogen atom, a phosphorus atom, or a trivalent group; and

L¹, L², L³, and L⁴ each independently represent a coordinating group or a hydrogen atom;

wherein at least one of L¹, L², L³, and L⁴ is a coordinating group represented by the following formula (2):

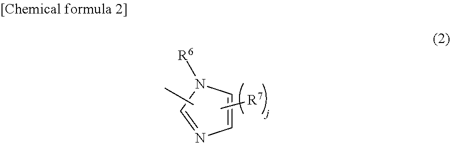

wherein
R⁶ represents a hydrogen atom or a substituent;
R⁷ represents a substituent; and
j represents an integer of from 0 to 2; and
when R⁶ and R⁷ each represent a substituent bonded to atoms adjacent to each other, R⁶ and R⁷ may be linked to form a ring; and
when j is 2 and two R⁷s each represent a substituent bonded to carbon atoms adjacent to each other, two R⁷s may be linked together to form a ring;
   or at least one of L¹, L², L³, and L⁴ is a coordinating group represented by the following formula (3):

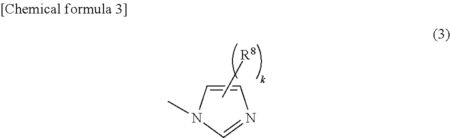

wherein
R⁸ represents a substituent; and
k is an integer of from 0 to 3; and
when k is 2 and R⁸s each represent a substituent bonded to carbon atoms adjacent to each other, R⁸s may be linked to form a ring; and
when k is 3, R⁸ bonded to the carbon atom at position 4 and R⁸ bonded to the carbon atom at position 5 may be linked together to form a ring.
   [5] The metal complex according to any of [1] to [4], wherein said metal complex is represented by the following composition formula (4):

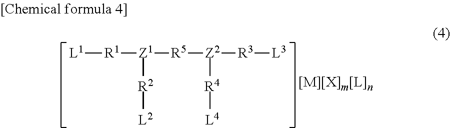

wherein

M represents an ion of a metal selected from the group consisting of cerium, praseodymium, ytterbium, and lutetium,

X represents a counter ion;

L represents a ligand having denticity of 4 or less; and

m is an integer of from 0 to 4, and n is an integer of 0 or more.

[6] The metal complex according to [4] or [5], wherein R¹, R², R³, R⁴, and R⁵ in said polydentate ligand each independently represent a divalent group represented by the following formula (5):

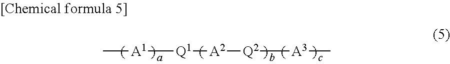

wherein

Q¹ and Q² each independently represent a divalent hydrocarbyl group that is optionally substituted, or a divalent heterocyclyl group that is optionally substituted; and

A¹, A², and A³ each independently represent a group represented by the following formula:

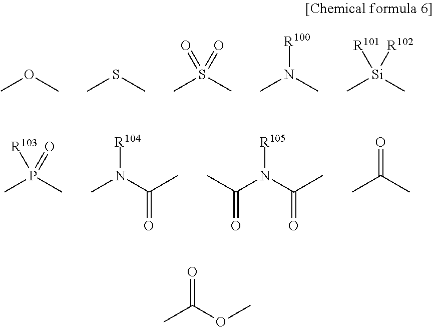

wherein
R¹⁰⁰, R¹⁰⁴, and R¹⁰⁵ each represent a hydrocarbyl group that is optionally substituted;
R¹⁰¹ and R¹⁰² each independently represent a hydrocarbyl group that is optionally substituted, or a hydrocarbyloxy group that is optionally substituted;
R¹⁰³ represents a hydrocarbyl group that is optionally substituted, or a hydrocarbyloxy group that is optionally substituted; and
a and c are each independently 0 or 1, and b is an integer of from 0 to 10.
   [7] The metal complex according to any of [4] to [6], wherein R¹, R², R³, and R⁴ in said polydentate ligand each independently represent a divalent hydrocarbyl group that is optionally substituted.
   [8] The metal complex according to any of [4] to [7], wherein Z¹ and Z² in said polydentate ligand are each a nitrogen atom.
   [9] The metal complex according to any of [4] to [8], wherein said polydentate ligand is represented by the following formula (6):

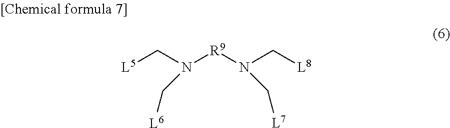

wherein

R⁹ represents a divalent group; and

L⁵, L⁶, L⁷, and L⁸ each independently represent a coordinating group or a hydrogen atom;

wherein at least one of L⁵, L⁶, L⁷, and L⁸ is said coordinating group represented by formula (2) or (3).

[10] The metal complex according to any of [4] to [9], wherein L¹, L², L³, and L⁴ in said polydentate ligand are each independently said coordinating group represented by formula (2) or (3).
[11] The metal complex according to any of [4] to [10], wherein said polydentate ligand is represented by the following formula (7):

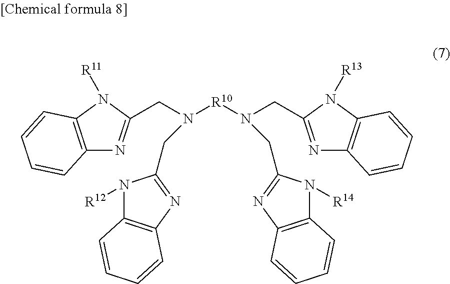

wherein

R¹⁰ represents a divalent group; and

R¹¹, R¹², R¹³, and R¹⁴ each independently represent a hydrogen atom or a substituent.

[12] The metal complex according to any of [1] to [11], wherein said metal complex is represented by the following composition formula (8):

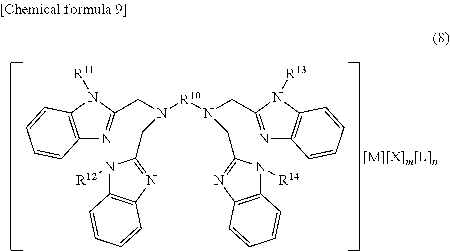

wherein

R¹⁵ represents a divalent group;

R¹⁶, R¹⁷, R¹⁸, and R¹⁹ each independently represent a hydrogen atom or a substituent;

M represents an ion of a metal selected from the group consisting of cerium, praseodymium, ytterbium, and lutetium;

X represents a counter ion;

L represents a ligand having denticity of 4 or less; and

m is an integer of from 0 to 4, and n is an integer of 0 or more.

[13] The metal complex according to any of [1] to [12], wherein said metal is cerium.
[14] A composition comprising the metal complex according to any of [1] to [13] and a charge transport material.
[15] An organic thin film obtained by using the metal complex according to any of [1] to [13] or the composition according to [14].
[16] A device obtained by using the metal complex according to any of [1] to [13], the composition according to [14], or the organic thin film according to [15].

### Effects of the Invention

The metal complex of the present invention is useful as a light-emitting material having excellent durability, since it has high durability against increases in temperature. Also, the metal complex of the present invention can have the advantageous effect of a high emission quantum yield, since it comprises a metal that can emit light based on 4f-5d transition. Further, the metal complex of the present invention can be preferably applied in the production of a device by a coating method, since it can achieve excellent solubility in an organic solvent.

### BRIEF DESCRIPTION OF DRAWINGS

FIG. 1 illustrates the emission spectra of the metal complexes (C-1) and (C-2).

FIG. 2 illustrates the fitting results of the emission spectrum of the metal complex (C-2).

### EMBODIMENT FOR CARRYING OUT THE INVENTION

### <Metal Complex>

The metal complex of the present invention comprises (a) a polydentate ligand having denticity of five or more that includes a heteroaromatic ring which contains two or more atoms selected from the group consisting of a nitrogen atom, an oxygen atom, and a sulfur atom, and (b) an ion of a metal selected from the group consisting of cerium, praseodymium, ytterbium, and lutetium. It is preferable that these metal ions have a valency of three.

Examples of the central metal comprised in the metal complex of the present invention may include cerium, praseodymium, ytterbium, and lutetium, which can exhibit luminescence based on 4f-5d transition. The central metal is preferably cerium or praseodymium, and more preferably cerium.

The polydentate ligand comprised in the metal complex of the present invention includes a heteroaromatic ring which contains two or more atoms selected from the group consisting of a nitrogen atom, an oxygen atom, and a sulfur atom. It is preferable that this heteroaromatic ring contains one or more nitrogen atoms or oxygen atoms having a lone electron pair that can coordinate with the metal as an essential ring-constituting atom. It is preferable that the number of nitrogen atoms and that of oxygen atoms in the heteroaromatic ring is each independently one, two, three, or four.

In one embodiment, the above-described heteroaromatic ring is an imidazole ring or a condensed imidazole ring. Examples of the condensed imidazole ring may include benzimidazole.

In another embodiment, examples of the above-described heteroaromatic ring may include a heteroaromatic ring represented by formulae (A-1) to (A-14), and a ring in which two or more heteroaromatic rings of such heteroaromatic rings are connected or condensed together.

The heteroaromatic ring is preferably a ring represented by any of formulae (A-1) to (A-10), more preferably a ring represented by formula (A-1), (A-3), (A-4), (A-7), (A-9), or (A-10), and still more preferably a ring represented by formula (A-1) or (A-7).

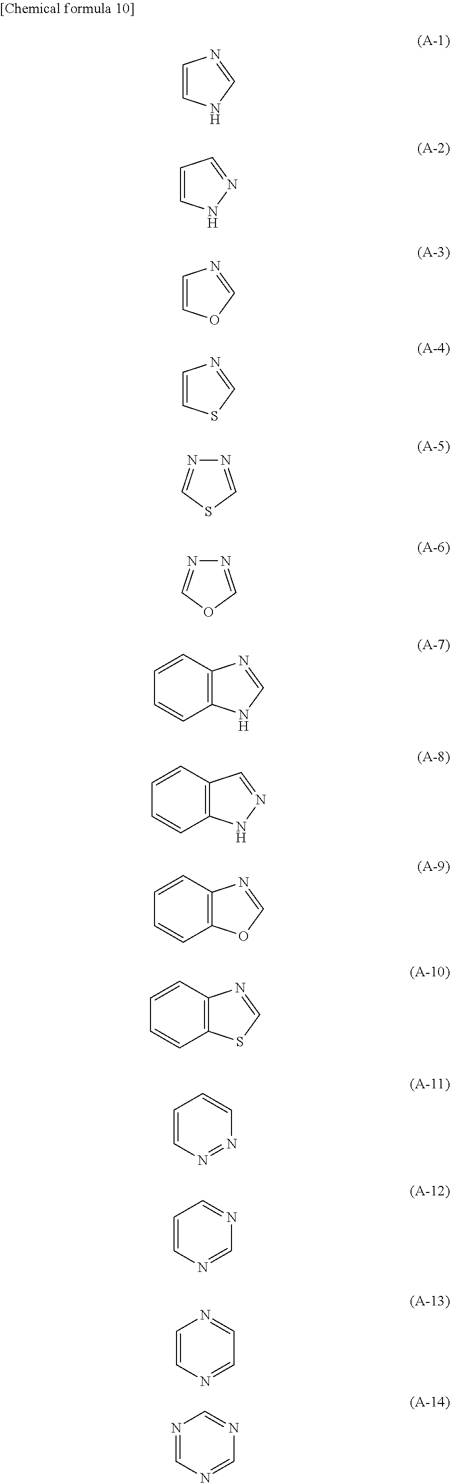

In the above-described imidazole ring or condensed imidazole ring, or the above-described heteroaromatic ring represented by any of formulae (A-1) to (A-14), a hydrogen atoms on the ring may be substituted with a hydrocarbyl group that is optionally substituted, a hydrocarbyloxy group that is optionally substituted, a hydrocarbylthio group that is optionally substituted, a heterocyclyl group that is optionally substituted, a halogen atom, a cyano group, an amido group that is optionally substituted, an imido group that is optionally substituted, a silyl group that is optionally substituted, an acyl group that is optionally substituted, an alkoxycarbonyl group that is optionally substituted, an alkoxysulfonyl group that is optionally substituted, an alkoxyphosphoryl group that is optionally substituted, a phosphino group that is optionally substituted, a phosphine oxide group that is optionally substituted, an amino group that is optionally substituted, a hydroxyl group, a mercapto group, a carboxyl group, a sulfo group, a phosphoric acid group, a phosphorous acid group, or a nitro group, or with an anionic group in which a hydrogen atom is removed from an amino group that is optionally substituted, a hydroxyl group, a mercapto group, a carboxyl group, a sulfo group, a phosphoric acid group, or a phosphorous acid group. If the substituents for the hydrogen atoms in the heteroaromatic ring are bonded to carbon atoms adjacent to each other, the substituents may be linked together to form a ring.

The substituent in the above-described imidazole ring or condensed imidazole ring or in the above-described formulae (A-1) to (A-14) is preferably a hydrocarbyl group that is optionally substituted, a hydrocarbyloxy group that is optionally substituted, a heterocyclyl group that is optionally substituted, a phosphine oxide group that is optionally substituted, a hydroxyl group, a carboxyl group, a sulfo group, or a phosphoric acid group, or an anionic group in which a hydrogen atom is removed from a hydroxyl group, a carboxyl group, a sulfo group, or a phosphoric acid group; more preferably a hydrocarbyl group that is optionally substituted, a hydrocarbyloxy group that is optionally substituted, a hydroxyl group, a carboxyl group, a sulfo group, or a phosphoric acid group; and still more preferably a hydrocarbyl group that is optionally substituted, or a hydrocarbyloxy group that is optionally substituted.

The hydrocarbyl group may be any of a straight chain, a branched chain or a cyclic structure, which usually has 1 to 30 carbon atoms, and preferably 1 to 12 carbon atoms. Examples of such a hydrocarbyl group may include a methyl group, an ethyl group, a 1-propyl group, a 2-propyl group, a 1-butyl group, a 2-butyl group, a sec-butyl group, a tert-butyl group, a pentyl group, a hexyl group, an octyl group, a decyl group, a dodecyl group, a 2-ethylhexyl group, a 3,7-dimethyloctyl group, a cyclopropyl group, a cyclopentyl group, a cyclohexyl group, a 1-adamantyl group, a 2-adamantyl group, a norbornyl group, an ammoniumethyl group, a benzyl group, an α,α-dimethylbenzyl group, a 1-phenethyl group, a 2-phenethyl group, a vinyl group, a propenyl group, a butenyl group, an oleyl group, an eicosapentaenyl group, a docosahexaenyl group, a 2,2-diphenylvinyl group, a 1,2,2-triphenylvinyl group, a 2-phenyl-2-propenyl group, a phenyl group, a 2-tolyl group, a 4-tolyl group, a 4-trifluoromethylphenyl group, a 4-methoxyphenyl group, a 4-cyanophenyl group, a 2-biphenylyl group, a 3-biphenylyl group, a 4-biphenylyl group, a terphenylyl group, a 3,5-diphenylphenyl group, a 3,4-diphenylphenyl group, a pentaphenylphenyl group, a 4-(2,2-diphenylvinyl)phenyl group, a 4-(1,2,2-triphenylvinyl)phenyl group, a fluorenyl group, a 1-naphthyl group, a 2-naphthyl group, a 9-anthryl group, a 2-anthryl group, a 9-phenanthryl group, 1-pyrenyl group, a chrysenyl group, a naphthacenyl group, and a coronyl group. The hydrocarbyl group is preferably a methyl group, an ethyl group, a 1-propyl group, a 2-propyl group, a 1-butyl group, a 2-butyl group, a sec-butyl group, a tert-butyl group, a pentyl group, a hexyl group, an octyl group, a decyl group, a dodecyl group, a 2-ethylhexyl group, a 3,7-dimethyloctyl group, a benzyl group, an α,α-dimethylbenzyl group, a 1-phenethyl group, a 2-phenethyl group, a vinyl group, a propenyl group, a butenyl group, a phenyl group, a 2-tolyl group, a 4-tolyl group, a 4-trifluoromethylphenyl group, a 4-methoxyphenyl group, a 2-biphenylyl group, a 3-biphenylyl group, a 4-biphenylyl group, a terphenylyl group, a fluorenyl group, a 1-naphthyl group, or a 2-naphthyl group, more preferably a methyl group, an ethyl group, a tert-butyl group, a 1-propyl group, a 1-butyl group, a 2-butyl group, a sec-butyl group, a tert-butyl group, a pentyl group, a hexyl group, an octyl group, a decyl group, a dodecyl group, a 2-ethylhexyl group, a benzyl group, an α,α-dimethylbenzyl group, a vinyl group, a butenyl group, a phenyl group, a 2-tolyl group, or a 4-tolyl group, still more preferably a methyl group, an ethyl group, a 1-propyl group, a hexyl group, or a vinyl group, and particularly preferably a methyl group, an ethyl group, or a 1-propyl group.

The hydrocarbyloxy group may be any of a straight chain, a branched chain or a cyclic structure, which usually has 1 to 30 carbon atoms, and preferably 1 to 12 carbon atoms. Examples of such a hydrocarbyloxy group may include a methoxy group, an ethoxy group, a 1-propyloxy group, a 2-propyloxy group, a 1-butyloxy group, a 2-butyloxy group, a sec-butyloxy group, a tert-butyloxy group, a pentyloxy group, a hexyloxy group, an octyloxy group, a decyloxy group, a dodecyloxy group, a 2-ethylhexyloxy group, a 3,7-dimethyloctyloxy group, a cyclopropyloxy group, a cyclopenthyloxy group, a cyclohexyloxy group, a 1-adamantyloxy group, a 2-adamantyloxy group, a norbornyloxy group, an ammoniumethoxy group, a trifluoromethoxy group, a benzyloxy group, an α,α-dimethylbenzyloxy group, a 2-phenethyloxy group, a 1-phenethyloxy group, a phenoxy group, a methoxyphenoxy group, an octylphenoxy group, a 1-naphthyloxy group, a 2-naphthyloxy group, and a pentafluorophenyloxy group. The hydrocarbyloxy group is preferably a methoxy group, an ethoxy group, a 1-propyloxy group, a 2-propyloxy group, a 1-butyloxy group, a 2-butyloxy group, a sec-butyloxy group, a tert-butyloxy group, a pentyloxy group, a hexyloxy group, an octyloxy group, a decyloxy group, a dodecyloxy group, a 2-ethylhexyloxy group, or a 3,7-dimethyloctyloxy group, and more preferably a methoxy group, an ethoxy group, or a 1-propyloxy group.

The hydrocarbylthio group may be any of a straight chain, a branched chain or a cyclic structure, which usually has 1 to 30 carbon atoms, and preferably 1 to 12 carbon atoms. Examples of such a hydrocarbylthio group may include a methylthio group, an ethylthio group, a 1-propylthio group, a 2-propylthio group, a 1-butylthio group, a 2-butylthio group, a sec-butylthio group, a tert-butylthio group, a pentylthio group, a hexylthio group, an octylthio group, a decylthio group, a dodecylthio group, a 2-ethylhexylthio group, a 3,7-dimethyloctylthio group, a cyclopropylthio group, a cyclopentylthio group, a cyclohexylthio group, a 1-adamantylthio group, a 2-adamantylthio group, a norbornylthio group, an ammoniumethylthio group, a trifluoromethylthio group, a benzylthio group, an α,α-dimethylbenzylthio group, a 2-phenethylthio group, a 1-phenethylthio group, a phenylthio group, a methoxyphenylthio group, an octylphenylthio group, a 1-naphthylthio group, a 2-naphthylthio group, and a pentafluorophenylthio group. The hydrocarbylthio group is preferably a methylthio group, an ethylthio group, a 1-propylthio group, a 2-propylthio group, a 1-butylthio group, a 2-butylthio group, a sec-butylthio group, a pentylthio group, a hexylthio group, an octylthio group, a decylthio group, a dodecylthio group, a 2-ethylhexylthio group, or a 3,7-dimethyloctylthio group, and more preferably a methylthio group, an ethylthio group, or a 1-propylthio group.

Examples of the heterocyclyl group may include a piperidinyl group, a piperazinyl group, a furyl group, a thienyl group, a pyrrolyl group, an imidazolyl group, an oxazolyl group, a thiazolyl group, and a pyridyl group. The heterocyclyl group is preferably a furyl group, a thienyl group, a pyrrolyl group, an imidazolyl group, an oxazolyl group, a thiazolyl group, or a pyridyl group, more preferably a thienyl group, an imidazolyl group, an oxazolyl group, a thiazolyl group, or a pyridyl group, and still more preferably a pyridyl group.

Examples of the halogen atom may include a fluorine atom, a chlorine atom, a bromine atom, and an iodine atom. The halogen atom is preferably a fluorine atom or a chlorine atom.

The amido group usually has 1 to 20 carbon atoms, and preferably 1 to 12 carbon atoms. Examples of such an amido group may include a formamido group, an acetamido group, a propioamido group, a butyramido group, a benzamido group, a trifluoroacetamido group, a pentafluoro benzamido group, a diformamido group, a diacetoamido group, a dipropioamido group, a dibutyramido group, a dibenzamido group, a ditrifluoroacetamido group, and a dipentafluorobenzamido group. The amido group is preferably a formamido group, an acetamido group, a propioamido group, a benzamido group, or a benzamido group.

The imido group is a group obtained by removing a hydrogen atom bonded to the nitrogen atom on an imide. The imido group usually has 4 to 20 carbon atoms, and preferably 4 to 12 carbon atoms. Examples of such an imido group may include an N-succinimide group, an N-phthalimide group, and a benzophenone imido group. Preferred is an N-phthalimide group.

The silyl group is a silyl group that is optionally substituted with 1 to 3 groups selected from the group consisting of an alkyl group, an aryl group, and an arylalkyl group. Such a silyl group usually has 1 to 60 carbon atoms, and preferably 1 to 36 carbon atoms. Preferred examples of such a silyl group include a trimethylsilyl group, a triethylsilyl group, a tripropylsilyl group, a tri-1-propyl silyl group, a dimethyl-1-propylsilyl group, a diethyl-1-propylsilyl group, a t-butyldimethylsilyl group, a pentyldimethylsilyl group, a hexyldimethylsilyl group, a heptyldimethylsilyl group, an octyldimethylsilyl group, a 2-ethylhexyl-dimethylsilyl group, a nonyldimethylsilyl group, a decyldimethylsilyl group, a 3,7-dimethyloctyl-dimethylsilyl group, a lauryldimethylsilyl group, a triphenylsilyl group, a tri-p-xylylsilyl group, a tribenzylsilyl group, a diphenylmethylsilyl group, a t-butylphenylsilyl group, and a dimethylphenylsilyl group. The silyl group is more preferably a trimethylsilyl group, a triethylsilyl group, or a tripropylsilyl group, and still more preferably a trimethylsilyl group.

The acyl group usually has 1 to 20 carbon atoms, and preferably 1 to 12 carbon atoms. Examples of such an acyl group may include a formyl group, an acetyl group, a propionyl group, a butyryl group, an isobutyryl group, a pivaloyl group, a benzoyl group, a trifluoroacetyl group, and a pentafluorobenzoyl group. The acyl group is preferably an acetyl group or a benzoyl group.

The alkoxycarbonyl group usually has 1 to 20 carbon atoms, and preferably 1 to 12 carbon atoms. Examples of such an alkoxycarbonyl group may include a methoxycarbonyl group, an ethoxycarbonyl group, a propyloxycarbonyl group, an isopropyloxycarbonyl group, a butoxycarbonyl group, an isobutoxycarbonyl group, an s-butoxycarbonyl group, a t-butoxycarbonyl group, a pentyloxycarbonyl group, a hexyloxycarbonyl group, a heptyloxycarbonyl group, an octyloxycarbonyl group, a 2-ethylhexyloxycarbonyl group, a nonyloxycarbonyl group, a decyloxycarbonyl group, a 3,7-dimethyloctyloxy carbonyl group, and a dodecyloxycarbonyl group. The alkoxycarbonyl group is preferably a methoxycarbonyl group, an ethoxycarbonyl group, a propyloxycarbonyl group, an isopropyloxycarbonyl group, a butoxycarbonyl group, or an isobutoxycarbonyl group, and more preferably a methoxycarbonyl group or an ethoxycarbonyl group.

The alkoxysulfonyl group usually has 1 to 20 carbon atoms, and preferably 1 to 12 carbon atoms. Examples of such an alkoxysulfonyl group may include a methoxysulfonyl group, ethoxysulfonyl group, a propyloxysulfonyl group, an isopropyloxysulfonyl group, a butoxysulfonyl group, an isobutoxysulfonyl group, an s-butoxysulfonyl group, a t-butoxyulfonyl group, a pentyloxysulfonyl group, a hexyloxysulfonyl group, a heptyloxysulfonyl group, an octyloxysulfonyl group, a 2-ethylhexyloxysulfonyl group, a nonyloxysulfonyl group, a decyloxysulfonyl group, a 3,7-dimethyloctyloxysulfonyl group, and a dodecyloxysulfonyl group. The alkoxysulfonyl group is preferably a methoxysulfonyl group, an ethoxysulfonyl group, a propyloxysulfonyl group, an isopropyloxysulfonyl group, a butoxysulfonyl group, or an isobutoxysulfonyl group, and more preferably a methoxysulfonyl group or an ethoxysulfonyl group.

The alkoxyphosphoryl group usually has 1 to 20 carbon atoms, and preferably 1 to 12 carbon atoms. Examples of such an alkoxyphosphoryl group may include a dimethoxyphosphoryl group, a diethoxyphosphoryl group, a dipropoxyphosphoryl group, a diisopropoxyphosphoryl group, a dibutoxyphosphoryl group, and an ethylenedioxyphosphoryl group. The alkoxyphosphoryl group is preferably a dimethoxyphosphoryl group.

The phosphino group is a phosphino group that is optionally substituted with 1 or 2 groups selected from the group consisting of an alkyl group, an aryl group, and an arylalkyl group. Such a phosphino group usually has 1 to 20 carbon atoms, and preferably 1 to 12 carbon atoms. Examples of such a phosphino group may include a phenylphosphino group, a diphenylphospino group, a methylphosphino group, a dimethylphosphino group, an ethylphosphino group, a diethylphosphino group, a propylphosphino group, a dipropylphosphino group, a butylphosphino group, and a dibutylphosphino group. The phosphino group is preferably a diphenylphospino group, a dimethylphosphino group, a diethylphosphino group, a dipropylphosphino group, or a dibutylphosphino group, more preferably a diphenylphospino group or a dimethylphosphino group, and particularly preferably a diphenylphospino group.

The phosphine oxide group is a phosphine oxide group that is optionally substituted with 1 or 2 groups selected from the group consisting of an alkyl group, an aryl group, and an arylalkyl group. Such a phosphine oxide group usually has 1 to 20 carbon atoms, and preferably 1 to 12 carbon atoms. Examples of such a phosphine oxide group may include a phenylphosphine oxide group, a diphenylphosphine oxide group, a methylphosphine oxide group, a dimethylphosphine oxide group, an ethylphosphine oxide group, a diethylphosphine oxide group, a propylphosphine oxide group, a dipropylphosphine oxide group, a butylphosphine oxide group, and a dibutylphosphine oxide group. The phosphine oxide group is preferably a diphenylphosphine oxide group, a dimethylphosphine oxide group, a diethylphosphine oxide group, a dipropylphosphine oxide group, or a dibutylphosphine oxide group, more preferably a diphenylphosphine oxide group or a dimethylphosphine oxide group, and particularly preferably a diphenylphosphine oxide group.

The amino group is an amino group that is substituted with 1 to 3 groups selected from the group consisting of an alkyl group, an aryl group, and an arylalkyl group, or —NH₂. Such an amino group usually has 1 to 60 carbon atoms, and preferably 1 to 36 carbon atoms. Examples of this amino group may include a phenylamino group, a diphenylamino group, a methylamino group, a dimethylamino group, an ethylamino group, a diethylamino group, a propylamino group, a dipropylamino group, a butylamino group, and a dibutylamino group. The amino group is preferably a diphenylamino group, a methylamino group, a dimethylamino group, an ethylamino group, a diethylamino group, a dipropylamino group, or a dibutylamino group, and more preferably a methylamino group, an ethylamino group, or a diphenylamino group.

The anionic group in which a hydrogen atom is removed from an amino group that is optionally substituted, a hydroxyl group, a mercapto group, a carboxyl group, a sulfo group, a phosphoric acid group, or a phosphorous acid group may have a counter ion. Examples of the counter ion may include a lithium ion, a sodium ion, a potassium ion, a rubidium ion, a cesium ion, and an ammonium ion. The counter ion is preferably a sodium ion, a potassium ion, or an ammonium ion.

Examples of the substituent that the above-described hydrocarbyl group, hydrocarbyloxy group, hydrocarbylthio group, heterocyclyl group, amido group, imido group, silyl group, acyl group, alkoxycarbonyl group, alkoxysulfonyl group, alkoxyphosphoryl group, phosphino group, phosphine oxide group, amino group, and anionic group in which a hydrogen atom is removed from an amino group may have (hereinafter, the term “substituent” in the present specification has the same meaning) may include a hydrocarbyl group, a hydrocarbyloxy group, a hydrocarbylthio group, a heterocyclyl group, a halogen atom, a cyano group, an amido group, an imido group, a silyl group, an acyl group, an alkoxycarbonyl group, an alkoxysulfonyl group, an alkoxyphosphoryl group, a phosphino group, a phosphine oxide group, an amino group, a hydroxyl group, a mercapto group, a carboxyl group, a sulfo group, a phosphoric acid group, a phosphorous acid group, and a nitro group, and an anionic group in which a hydrogen atom is removed from an amino group, a hydroxyl group, a mercapto group, a carboxyl group, a sulfo group, a phosphoric acid group, or a phosphorous acid group. The substituent is preferably a hydrocarbyl group, a hydrocarbyloxy group, a hydrocarbylthio group, a heterocyclyl group, a hydroxyl group, a carboxyl group, a sulfo group, or a phosphoric acid group, and more preferably a hydrocarbyl group. The specific examples and preferred examples of these groups are the same as the groups corresponding to the description of the above-described substituents in formulae (A-1) to (A-14). If the substituent is plurally present, they may be the same as or different from each other.

The number of heteroaromatic rings in the polydentate ligands is 1 or more, preferably 2 or more, more preferably 3 or more, and still more preferably 4 or more. Further, the number of heteroaromatic rings in the polydentate ligand is 12 or less, preferably 10 or less, more preferably 8 or less, and still more preferably 6 or less.

The number of polydentate ligands in the metal complex of the present invention is usually 1 to 3, preferably 1 or 2, and more preferably 1.

The denticity of the polydentate ligands is 5 or more, preferably 5 to 12, more preferably 6 to 10, and still more preferably 6 to 8.

In addition to the above-described heteroaromatic ring that contains a nitrogen atom or an oxygen atom that can be coordinated to a metal, the polydentate ligands may include an atom having a lone electron pair that can be coordinated to a metal, which is not on the heteroaromatic ring. Examples of such an atom may include a nitrogen atom and an oxygen atom. The number of such an atom is 1 or more, preferably 2 or more, and more preferably 3 or more. Further, the number of such an atom is 11 or less, preferably 9 or less, more preferably 7 or less, and still more preferably 5 or less.

In one embodiment, the polydentate ligands is represented by the following formula (1).

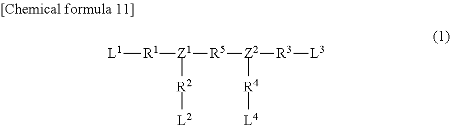

R¹, R², R³, R⁴ and R⁵ each independently represent a divalent group or a direct bond. Examples of such a divalent group may include a group represented by the following formula:

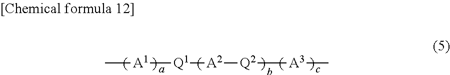

wherein Q¹ and Q² each independently represent a divalent hydrocarbyl group that is optionally substituted, or a divalent heterocyclyl group that is optionally substituted; and A¹, A², and A³ each independently represent a group represented by the following formulae (Z1) to (Z10):

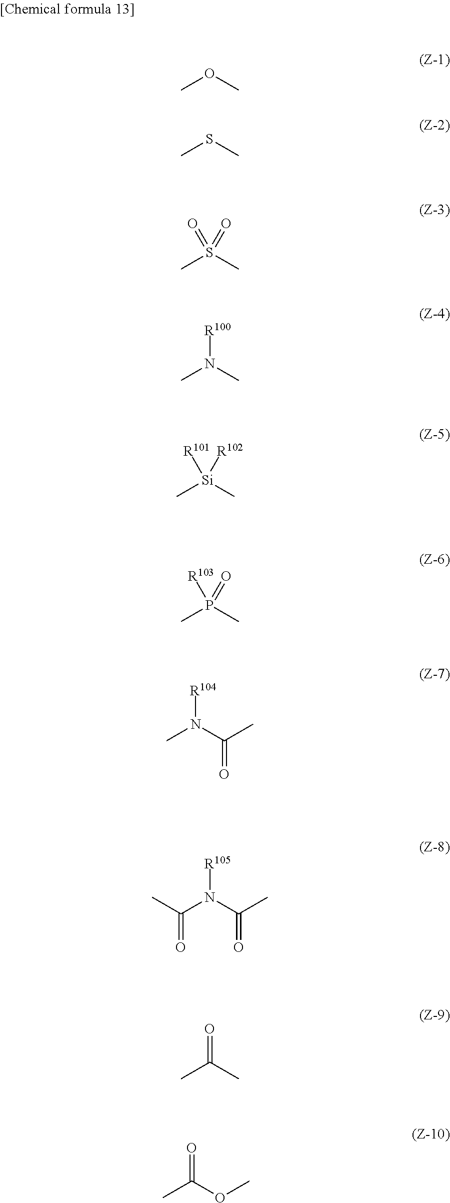

wherein R¹⁰⁰, R¹⁰⁴, and R¹⁰⁵ represent a hydrocarbyl group that is optionally substituted; R¹⁰¹ and R¹⁰² each independently represent a hydrocarbyl group that is optionally substituted, or a hydrocarbyloxy group that is optionally substituted; and R¹⁰³ represents a hydrocarbyl group that is optionally substituted, or a hydrocarbyloxy group that is optionally substituted. The specific examples and preferred examples of these hydrocarbyl groups and hydrocarbyloxy groups are the same as the groups corresponding to the description of the above-described substituents in formulae (A-1) to (A-14).

A¹, A², and A³ are preferably the above-described groups represented by formulae (Z-1) to (Z-6), more preferably the above-described groups represented by formulae (Z-1) to (Z-4), still more preferably the above-described groups represented by formulae (Z-1), (Z-2), or (Z-4), and particularly preferably the above-described group represented by formula (Z-1).

a and c are each independently an integer of 0 or 1, and preferably 0. b is an integer of from 0 to 10, preferably an integer of from 0 to 5, more preferably an integer of from 0 to 3, and still more preferably an integer of from 0 to 2.

The divalent hydrocarbyl group and divalent heterocyclyl group in Q¹ and Q² are divalent groups produced by removing one hydrogen atom from the above-described hydrocarbyl group and heterocyclyl group, respectively. The specific examples and preferred examples of these divalent groups are the same as the groups corresponding to the description of the above-described substituents in formulae (A-1) to (A-14), except for the point of removing one hydrogen atom.

Examples of the divalent group of R¹, R², R³, R⁴, and R⁵ may include a divalent hydrocarbyl group that is optionally substituted, a divalent hydrocarbyloxy group that is optionally substituted, a divalent hydrocarbylthio group that is optionally substituted, a divalent heterocyclyl group that is optionally substituted, a divalent amido group that is optionally substituted, a divalent imido group that is optionally substituted, a divalent silyl group that is optionally substituted, a divalent acyl group that is optionally substituted, a divalent alkoxycarbonyl group that is optionally substituted, a divalent alkoxysulfonyl group that is optionally substituted, a divalent alkoxyphosphoryl group that is optionally substituted, and a divalent amino group that is optionally substituted. The divalent group is preferably a divalent hydrocarbyl group that is optionally substituted, a divalent hydrocarbyloxy group that is optionally substituted, a divalent hydrocarbylthio group that is optionally substituted, a divalent heterocyclyl group that is optionally substituted, a divalent silyl group that is optionally substituted, a divalent alkoxycarbonyl group that is optionally substituted, or a divalent amino group that is optionally substituted, and more preferably a divalent hydrocarbyl group that is optionally substituted.

The divalent hydrocarbyl group, divalent hydrocarbyloxy group, divalent hydrocarbylthio group, divalent heterocyclyl group, divalent amido group, divalent imido group, divalent silyl group, divalent acyl group, divalent alkoxycarbonyl group, divalent alkoxysulfonyl group, divalent alkoxyphosphoryl group, and divalent amino group are divalent groups produced by removing one hydrogen atom from the aforementioned hydrocarbyl group, hydrocarbyloxy group, hydrocarbylthio group, heterocyclyl group, amido group, imido group, silyl group, acyl group, alkoxycarbonyl group, alkoxysulfonyl group, alkoxyphosphoryl group, and amino group, respectively. The specific examples and preferred examples of these divalent groups are the same as the groups corresponding to the description of the above-described substituents in formulae (A-1) to (A-14), except for the point of removing one hydrogen atom.

Z¹ and Z² each independently represent a nitrogen atom, a phosphorus atom, or a trivalent group. Examples of such a trivalent group may include a trivalent hydrocarbyl group that is optionally substituted. Z¹ and Z² are preferably a nitrogen atom or a phosphorus atom, and more preferably a nitrogen atom. The trivalent hydrocarbyl group and the like are a trivalent group produced by removing two hydrogen atoms from the aforementioned hydrocarbyl group and the like. The specific examples and preferred examples of the trivalent hydrocarbyl group are the same as described for the hydrocarbyl group in the description of the above-described substituents in formulae (A-1) to (A-14), except for the point of removing two hydrogen atoms.

L¹, L², L³, and L⁴ each independently represent a coordinating group or a hydrogen atom. The coordinating group is a group that contains one or more nitrogen atoms or oxygen atoms having a lone electron pair that can be coordinated to a metal. Examples of such a coordinating group may include a hydrocarbyloxy group that is optionally substituted, a heterocyclyl group that is optionally substituted, an amido group that is optionally substituted, an acyl group that is optionally substituted, an alkoxycarbonyl group that is optionally substituted, a phosphine oxide group that is optionally substituted, an amino group that is optionally substituted, a hydroxyl group, a carboxyl group, a sulfo group, a phosphoric acid group, and a nitro group, and an anionic group in which a hydrogen atom is removed from a hydroxyl group, a carboxyl group, a sulfo group, or a phosphoric acid group. The coordinating group is preferably a heterocyclyl group that is optionally substituted, a phosphine oxide group that is optionally substituted, an amino group that is optionally substituted, a hydroxyl group, a carboxyl group, a sulfo group, or a phosphoric acid group, or an anionic group in which a hydrogen atom is removed from a hydroxyl group, a carboxyl group, a sulfo group, or a phosphoric acid group, more preferably a heterocyclyl group that is optionally substituted or an anionic group in which a hydrogen atom is removed from a carboxyl group, a sulfo group, or a phosphoric acid group, and still more preferably a heterocyclyl group that is optionally substituted.

The specific examples and preferred examples of the hydrocarbyloxy group, amido group, acyl group, alkoxycarbonyl group, alkoxysulfonyl group, alkoxyphosphoryl group, and phosphine oxide group, which are examples of the coordinating groups for L¹, L², L³, and L⁴, are the same as described for the groups corresponding to the description of the above-described substituents in formulae (A-1) to (A-14).

Examples of the heterocyclyl group, which is an example of a coordinating group for L¹, L², L³, and L⁴, may include a pyridyl group, a quinolyl group, a pyrimidyl group, a pyrazinyl group, a pyrazolyl group, an imidazolyl group, an oxazolyl group, a thiazolyl group, a benzimidazolyl group, a benzoxazolyl group, a triazinyl group, a pyrimidinyl group, a pyrazinyl group, a bipyridinyl-group, a biquinolyl group, a terpyridyl group, and a phenanthrolinyl group. The heterocyclyl group is preferably a pyridyl group, a quinolyl group, an imidazolyl group, an oxazolyl group, a thiazolyl group, a benzimidazolyl group, a benzoxazolyl group, or a triazinyl group, more preferably a pyridyl group, a quinolyl group, an imidazolyl group, or a benzimidazolyl group, still more preferably an imidazolyl group or a benzimidazolyl group, and particularly preferably a benzimidazolyl group.

Examples of the amino group, which is an example of a coordinating group for L¹, L², L³, and L⁴, may include a phenylamino group, a diphenylamino group, a methylamino group, a dimethylamino group, an ethylamino group, a diethylamino group, a propylamino group, a dipropylamino group, a butylamino group, and a dibutylamino group. The amino group is preferably a phenylamino group, a methylamino group, an ethylamino group, a propylamino group, or a butylamino group, and more preferably a phenylamino group.

The anionic group in which a hydrogen atom is removed from an amino group that is optionally substituted, a hydroxyl group, a carboxyl group, a sulfo group, a phosphoric acid group, or a phosphorous acid group, which is an example of a coordinating group for L¹, L², L³, and L⁴, may have a counter ion. Examples of the counter ion may include a lithium ion, a sodium ion, a potassium ion, a rubidium ion, a cesium ion, and an ammonium ion. The counter ion is preferably a sodium ion, a potassium ion, or an ammonium ion.

At least one (i.e., one, two, three, or all) of L¹, L², L³, and L⁴ is a coordinating group represented by the following formula (2) or (3).

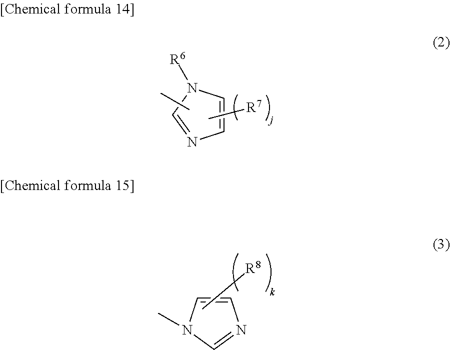

R⁶ represents a hydrogen atom or a substituent. When R⁶ represents a substituent, examples of R⁶ may include a hydrocarbyl group that is optionally substituted, a heterocyclyl group that is optionally substituted, a silyl group that is optionally substituted, and an acyl group that is optionally substituted. R⁶ preferably represents a hydrocarbyl group that is optionally substituted.

R⁷ represents a substituent, and j is an integer of from 0 to 2. Examples of R⁷ may include a hydrocarbyl group that is optionally substituted, a hydrocarbyloxy group that is optionally substituted, a heterocyclyl group that is optionally substituted, a hydroxyl group, a carboxyl group, a sulfo group, and a phosphoric acid group, and an anionic group in which a hydrogen atom is removed from a hydroxyl group, a carboxyl group, a sulfo group, or a phosphoric acid group. R⁷ is preferably a hydrocarbyl group that is optionally substituted, a hydrocarbyloxy group that is optionally substituted, a hydroxyl group, a carboxyl group, a sulfo group, or a phosphoric acid group, and more preferably a hydrocarbyl group that is optionally substituted. The specific examples and preferred examples of these groups are the same as the groups described above corresponding to the description of the above-described substituents in formulae (A-1) to (A-14). When j is 2, the two substituents may be the same or different from each other. Further, when j is 2 and two R⁷s each represent a substituent bonded to carbon atoms adjacent to each other, two R⁷s may be linked together to form a ring.

R⁸ represents a substituent, and k is an integer of from 0 to 3. Examples of R⁸ may include a hydrocarbyl group that is optionally substituted, a hydrocarbyloxy group that is optionally substituted, a heterocyclyl group that is optionally substituted, a hydroxyl group, a carboxyl group, a sulfo group, and a phosphoric acid group, and an anionic group in which a hydrogen atom is removed from a hydroxyl group, a carboxyl group, a sulfo group, or a phosphoric acid group. R⁸ is preferably a hydrocarbyl group that is optionally substituted, a hydrocarbyloxy group that is optionally substituted, a hydroxyl group, a carboxyl group, a sulfo group, or a phosphoric acid group, and more preferably a hydrocarbyl group that is optionally substituted. The specific examples and preferred examples of these groups are the same as the groups described above for the groups corresponding to the above-described substituents. When k is 2 or 3, the two or three substituents may be the same or different from each other. Further, when k is 2 and two R⁸s each represent substituents bonded to carbon atoms adjacent to each other, two R⁸s may be linked together to form a ring. When k is 3, R⁸ bonded to the carbon atom at position 4 and R⁸ bonded to the carbon atom at position 5 may be linked together to form a ring.

Examples of the above-described polydentate ligand may include the ligands represented by the following formulae (B-1) to (B-15). In these formulae, OH may also be an O⁻ obtained by dehydrogenation.

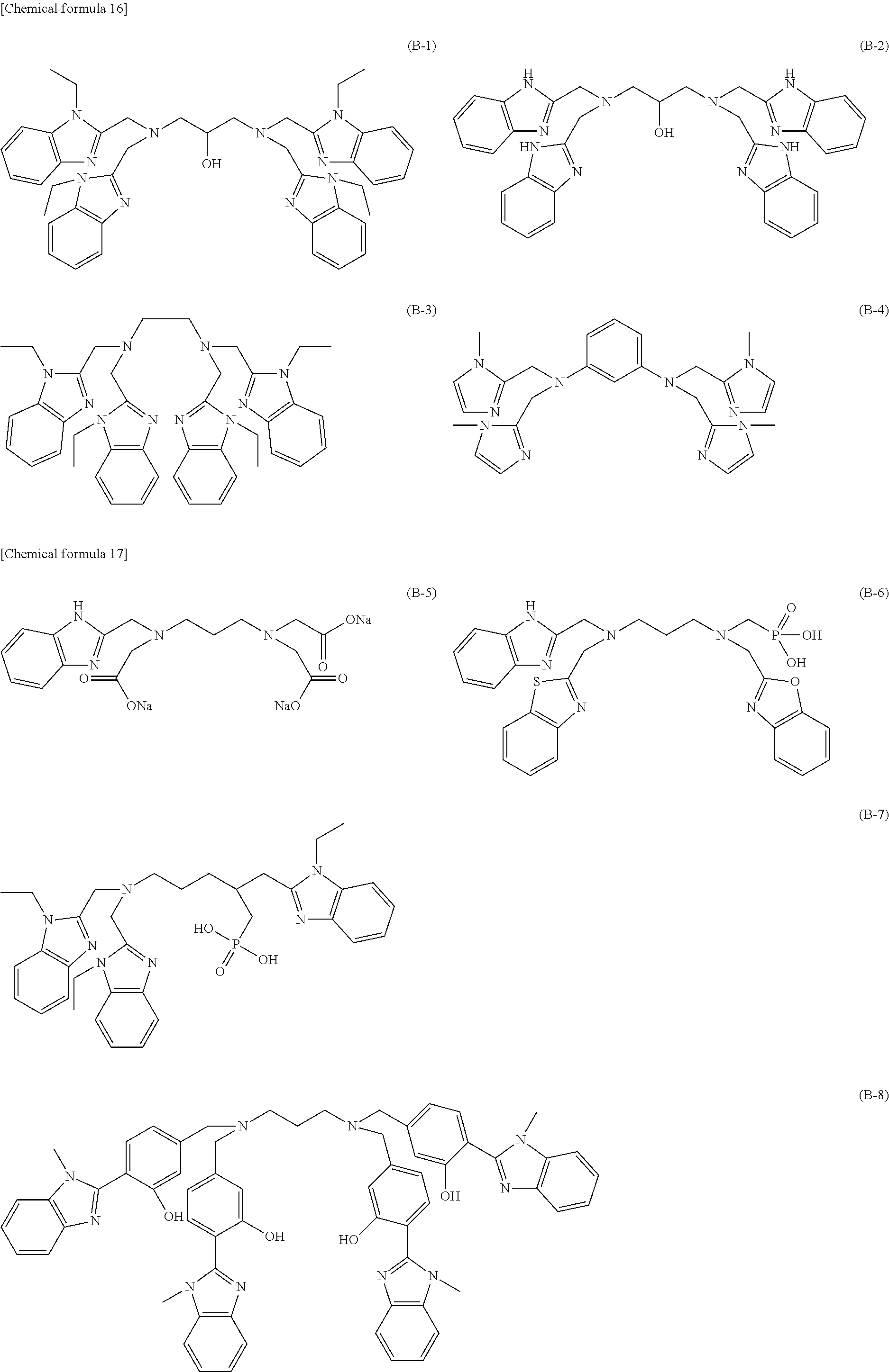

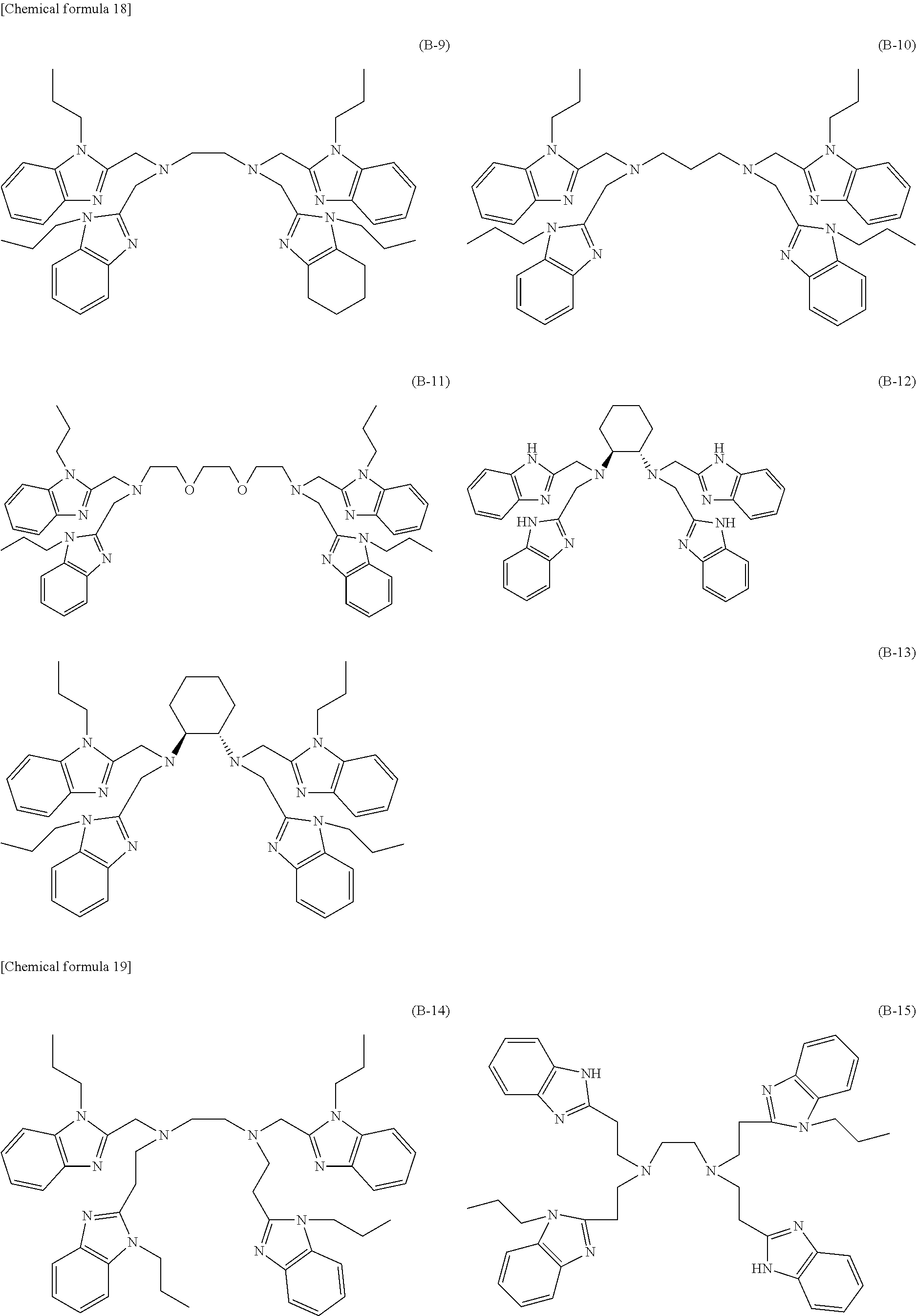

In addition to the polydentate ligand having denticity of five or more, the metal complex of the present invention may have one or a plurality of ligands (L) having denticity of four or less (for example, monodentate or bidentate) or a counter ion (X). Such a ligand is preferably an atom group that contains atoms selected from the group consisting of an oxygen atom, a nitrogen atom, and a phosphorus atom. Examples of such a ligand may include water, methanol, ethanol, acetone, tetrahydrofuran, dimethyl sulfoxide, triallyl phosphine oxide, trialkyl phosphine oxide, pyridine, quinoline, pyrazole, imidazole, oxazole, thiazole, benzimidazole, benzoxazole, benzothiazole, triazine, pyrimidine, pyrazine, bipyridine, biquinoline, terpyridine, phenanthroline, triallylphosphine, trialkylphosphine, and trialkylamine. Examples of the counter ion may include a fluoride ion, a chloride ion, a bromide ion, an iodide ion, a sulfate ion, a nitrate ion, a carbonate ion, an acetate ion, a perchlorate ion, a tetrafluoroborate ion, a hexafluorophosphate ion, a hexafluoroantimonate ion, a hexafluoroarsenate ion, a methanesulfonate ion, a trifluoromethanesulfonate ion, a trifluoroacetate ion, a benzenesulfonate ion, a para-toluenesulfonate ion, a dodecylbenzenesulfonate ion, a tetraphenylborate ion, and a tetrakis(pentafluorophenyl)borate ion. When L is an ionic ligand that is negatively charged, the counter ion may be a cation. Examples of such a cation may include a lithium ion, a sodium ion, a potassium ion, a rubidium ion, a cesium ion, and an ammonium ion. The counter ion is preferably a fluoride ion, a chloride ion, a nitrate ion, a perchlorate ion, a tetrafluoroborate ion, a hexafluorophosphate ion, a hexafluoroantimonate ion, a hexafluoroarsenate ion, a methanesulfonate ion, a trifluoromethanesulfonate ion, a trifluoroacetate ion, a benzenesulfonate ion, a para-toluenesulfonate ion, a dodecylbenzenesulfonate ion, a tetraphenylborate ion, or a tetrakis(pentafluorophenyl)borate ion. The counter ion is more preferably a chloride ion, a nitrate ion, a perchlorate ion, a tetrafluoroborate ion, a hexafluorophosphate ion, a methanesulfonate ion, a trifluoromethanesulfonate ion, a trifluoroacetate ion, a benzenesulfonate ion, a para-toluenesulfonate ion, a tetraphenylborate ion, or a tetrakis(pentafluorophenyl)borate ion, still more preferably a perchlorate ion, a tetrafluoroborate ion, a hexafluorophosphate ion, a trifluoromethanesulfonate ion, a trifluoroacetate ion, or a tetraphenylborate ion, and particularly preferably a tetrafluoroborate ion, a hexafluorophosphate ion, a trifluoromethanesulfonate ion, or a tetraphenylborate ion. Further, one kind or two or more kinds of these examples of X may be incorporated in one molecule.

Specifically, the metal complex of the present invention may be formed from the composition represented by the following composition formula (4).

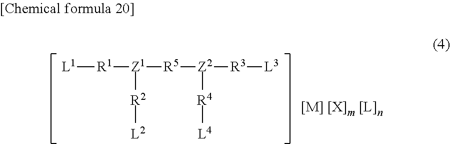

M represents an ion of a metal selected from the group consisting of cerium, praseodymium, ytterbium, and lutetium.

X represents a counter ion. This counter ion is the same as described above. m is an integer of from 0 to 4. m is preferably an integer of from 0 to 3, and more preferably 0 or 1.

L represents a ligand having denticity of 4 or less. This ligand having denticity of 4 or less is the same as that described above. n is an integer of 0 or more. n is preferably an integer of from 0 to 6, and more preferably an integer of from 0 to 3.

In another embodiment, the polydentate ligand is represented by the following formula (6).

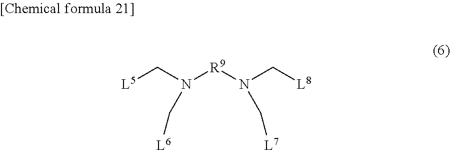

R⁹ represents a divalent group. The divalent group for R⁹ is the same as the divalent group described above for R⁵. The specific examples and preferred examples of R⁹ are the same as described above for R⁵.

L⁵, L⁶, L⁷, and L⁸ each independently represent a coordinating group or a hydrogen atom. The coordinating groups for L⁵, L⁶, L⁷, and L⁸ are the same as those for L¹, L², L³, and L⁴. The specific examples and preferred examples of L⁵, L⁶, L⁷, and L⁸ are the same as for L¹, L², L³, and L⁴. At least one (i.e., one, two, three, or all) of L⁵, L⁶, L⁷, and L⁸ is the above-described coordinating group represented by formula (2) or (3). Examples of the above-described polydentate ligands represented by formula (6) may include the above-described ligands represented by formulae (B-1) to (B-6) and (B-9) to (B-13).

In yet another embodiment, the polydentate ligand is represented by the following formula (7).

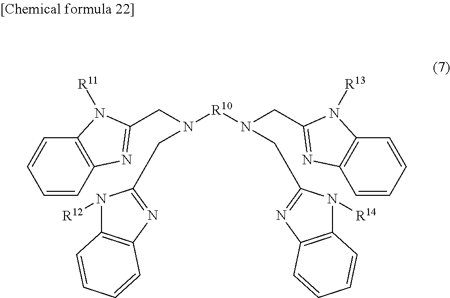

R¹⁰ represents a divalent group. The divalent group for R¹⁰ is the same as the divalent group described above for R⁹. The specific examples and preferred examples of R¹⁰ are the same as described above for R⁹.

R¹¹, R¹², R¹³, and R¹⁴ each independently represent a hydrogen atom or a substituent. The substituents for R¹¹, R¹², R¹³, and R¹⁴ are the same as the substituents described above for R⁶. The specific examples and preferred examples of R¹¹, R¹², R¹³, and R¹⁴ are the same as described above for R⁶. Examples of the polydentate ligands represented by formula (7) may include the above-described ligands represented by formulae (B-1) to (B-3) and (B-9) to (B-13).

The metal complex of the present invention may be formed from the composition represented by the following composition formula (8).

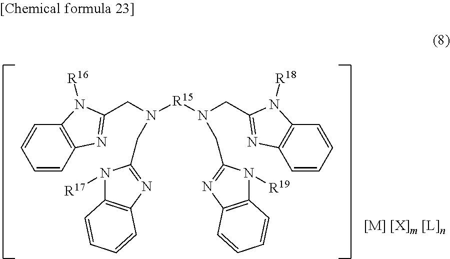

In the formula (8), R¹⁶ represents a divalent group. The divalent group for R¹⁵ is the same as the divalent group described above for R⁹. The specific examples and preferred examples of R¹⁵ are the same as described above for R⁹.

R¹⁶, R¹⁷, R¹⁸, and R¹⁹ each independently represent a hydrogen atom or a divalent group. The divalent groups for R¹⁶, R¹⁷, R¹⁸, and R¹⁹ are the same as the divalent group described above for R⁶. The specific examples and preferred examples of R¹⁶, R¹⁷, R¹⁸, and R¹⁹ are the same as described above for R⁶.

M represents an ion of a metal selected from the group consisting of cerium, praseodymium, ytterbium, and lutetium.

X represents a counter ion. The counter ion is the same as described above. m is an integer of from 0 to 4. m is preferably an integer of from 0 to 3, and more preferably 0 or 1.

L represents a ligand having denticity of 4 or less. The ligand having denticity of 4 or less is the same as described above. n is an integer of 0 or more. n is preferably an integer of from 0 to 6, and more preferably an integer of from 0 to 3.

More specifically, the metal complex of the present invention may be formed from the composition represented by the following formulae (C-1) to (C-13).

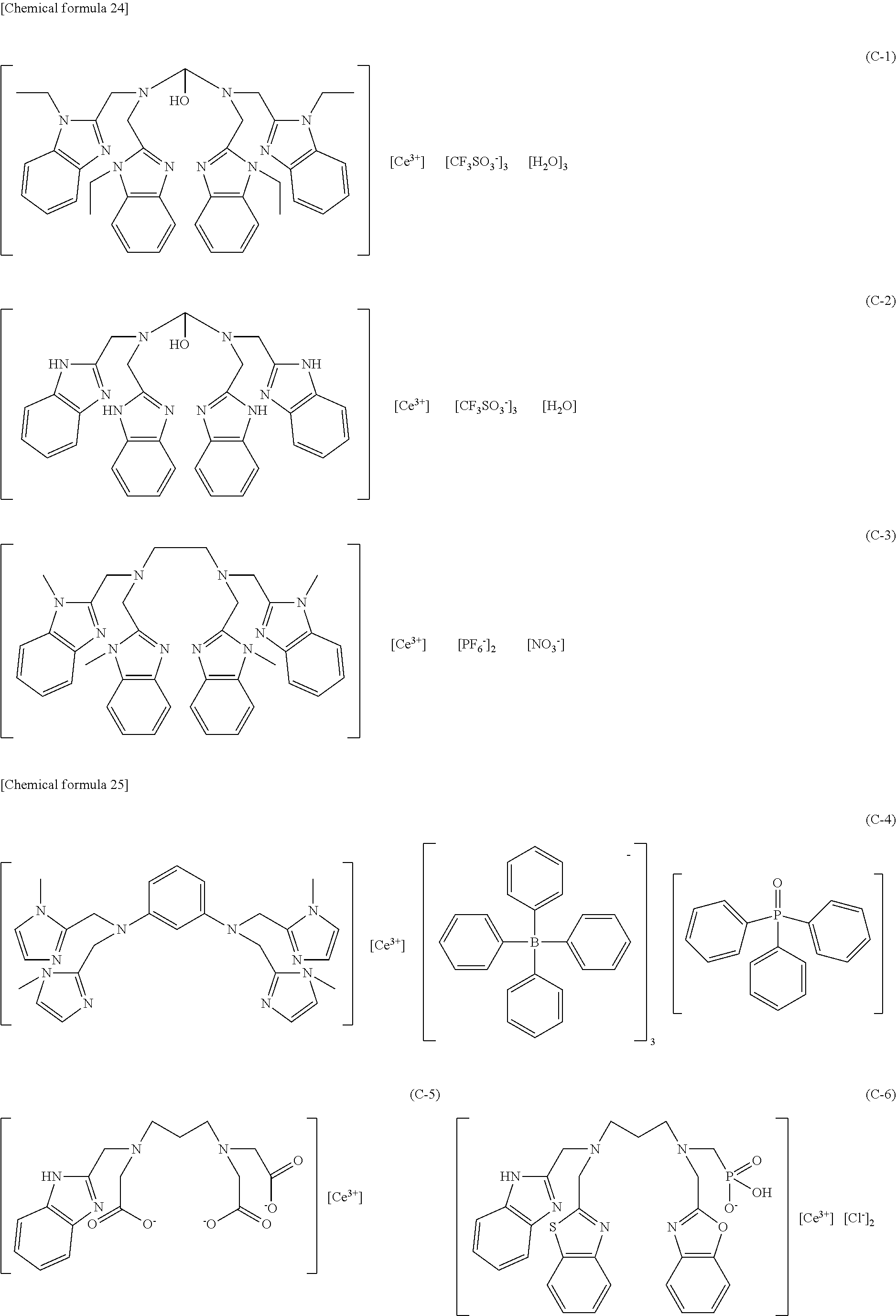

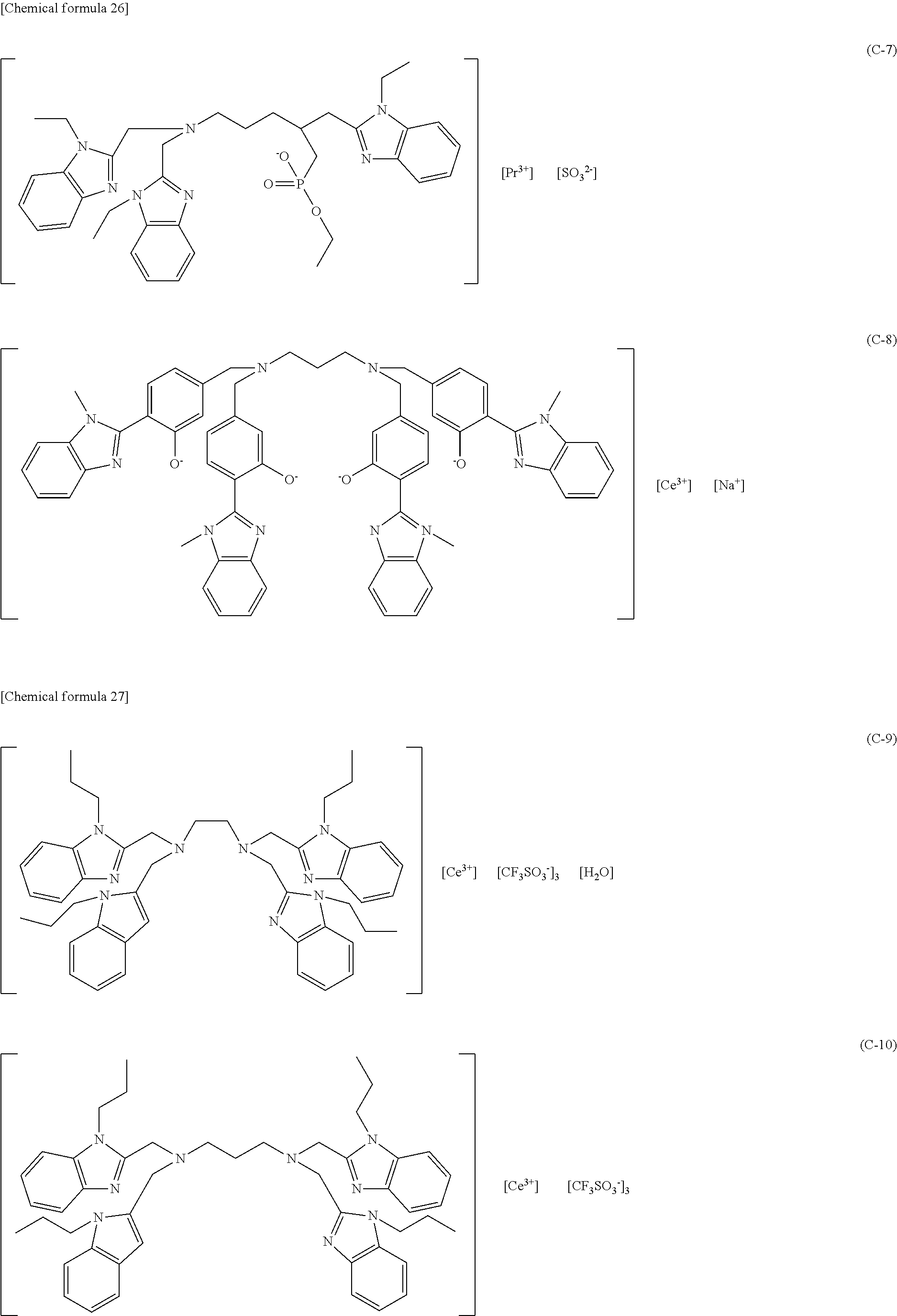

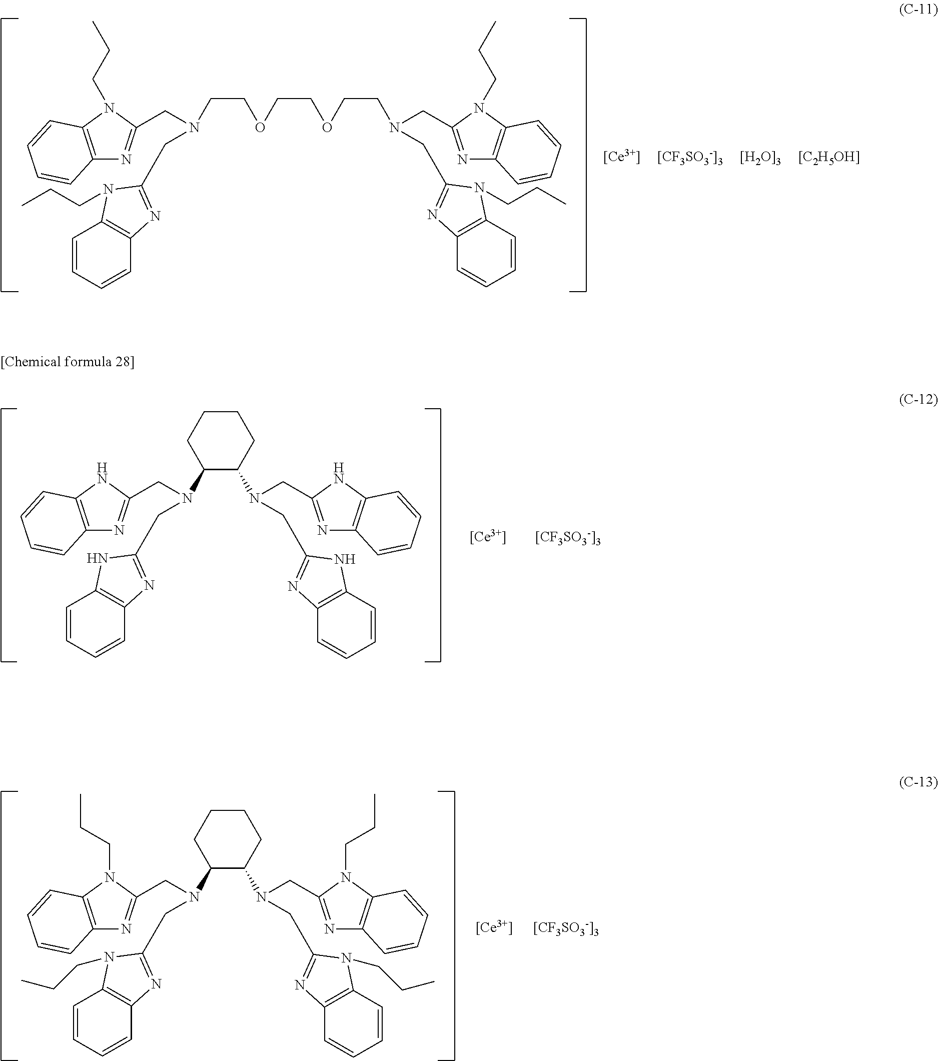

### <Method of Producing Metal Complex>

The metal complex of the present invention can be easily obtained by mixing a polydentate ligand and a metal salt (for example, cerium chloride(III) or cerium(III) trifluoromethanesulfonate) under room temperature in a solvent (for example, dichloromethane or acetonitrile), and collecting the obtained precipitate or evaporating the solvent in the obtained solution.

When performing the above mixing, to uniformly dissolve the polydentate ligand and metal complex in the solvent, or to facilitate stirring if the viscosity of the solution is high, a water-based solvent such as a buffer, or an organic solvent may be used, and an organic solvent is preferable.

Examples of the organic solvent may include a nitrile solvent such as acetonitrile and benzonitrile, a chlorinated solvent such as chloroform, methylene chloride, 1,2-dichloroethane, 1,1,2-trichloroethane, chlorobenzene, and o-dichlorobenzene, an ether solvent such as tetrahydrofuran and dioxane, an aromatic hydrocarbon solvent such as toluene and xylene, an aliphatic hydrocarbon solvent such as cyclohexane, methylcyclohexane, n-pentane, n-hexane, n-heptane, n-octane, n-nonane, and n-decane, a ketone solvent such as acetone, methyl ethyl ketone, and cyclohexanone, an ester solvent such as ethyl acetate, butyl acetate, and ethyl cellosolve acetate, a polyhydric alcohol solvent and derivatives thereof such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, and 1,2-hexandiol, an alcohol solvent such as methanol, ethanol, propanol, isopropanol, and cyclohexanol, a sulfoxide solvent such as dimethyl sulfoxide, and an amide solvent such as N-methyl-2-pyrrolidone and N,N-dimethylformamide. One kind of these organic solvents may be used, or two or more kinds may be used together.

### <Composition>

The composition of the present invention comprises the metal complex of the present invention and a charge transport material. The composition of the present invention is a liquid or a solid.

The charge transport material refers to a material that can be responsible for transporting a charge in a device such as an organic EL device, and examples of the charge transport material may include a hole transport material and an electron transport material. The charge transport material may be any of a low molecular compound, or a macromolecular compound or an oligomer. The macromolecular compound or oligomer is preferably conjugated.

As the hole transport material, materials that are known as hole transport materials for organic EL devices can be used, including a fluorene and derivatives thereof, an aromatic amine and derivatives thereof, carbazole derivatives, and polyparaphenylene derivatives. As the electron transport material, materials that are known as electron transport materials for organic EL devices can be used, including oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, and 8-hydroxyquinoline and metal complexes of derivatives thereof.

One kind of the metal complex of the present invention comprised in the composition may be used, or two or more kinds thereof may be used together. The content of the metal complex in the composition is preferably, based on 100 parts by mass of the charge transport material, 0.01 to 80 parts by mass, and more preferably 0.1 to 60 parts by mass. If the content of the metal complex is less than this lower limit, it tends to be difficult to obtain sufficient light emission from the metal complex. On the other hand, if the content exceeds the above upper limit, the emission intensity from the metal complex tends to weaken, and it tends to be more difficult to form a uniform film during thin film formation.

### <Organic Thin Film>

The organic thin film of the present invention uses the metal complex of the present invention or the composition of the present invention. The organic thin film of the present invention can be formed by, for example, a given film-formation method that uses the composition of the present invention in a liquid state. Examples of the organic thin film of the present invention may include a light-emitting thin film, a conductive thin film, and an organic semiconductor thin film. The thickness of the thin film is preferably 1 to 500 nm, and more preferably 5 to 200 nm.

### <Device>

The device of the present invention uses the metal complex of the present invention, the composition of the present invention, or the organic thin film of the present invention. Examples of the device of the present invention may include a light-emitting device, a switching device, and a photovoltaic device, which have a functional layer that comprises the composition of the present invention or the organic thin film of the present invention. Examples of the device include a device including a positive electrode, a functional layer comprising the metal complex of the present invention or the composition of the present invention which is disposed on this positive electrode, and a negative electrode that is disposed on this functional layer. More specifically, example of the device of the present invention include a device including a positive electrode, the organic thin film of the present invention which is a functional layer that is disposed on this positive electrode, and a negative electrode that is disposed on this organic thin film. The functional layer refers to a layer having a photoelectric function, that is, a thin film having a light-emitting property, a conductivity, and a photovoltaic function. Therefore, when the device of the present invention is a light-emitting device, the organic thin film that uses the metal complex of the present invention or the composition of the present invention corresponds to the light-emitting layer.

The device of the present invention may further include a charge transport layer or a charge block layer between the positive electrode and the negative electrode. The charge transport layer is a hole transport layer or an electron transport layer. The hole transport layer refers to a layer that has a function for transporting holes. The electron transport layer refers to a layer that has a function for transporting electrons. Further, the charge block layer refers to a hole block layer or an electron block layer. The hole block layer refers to a layer that has a function for transporting electrons and trapping holes transported from the positive electrode. The electron block layer refers to a layer that has a function for transporting holes and trapping electrons transported from the negative electrode.

Examples of the device of the present invention may include a device including an electron transport layer or a hole block layer between a negative electrode and a light-emitting layer, a device including a hole transport layer or an electron block layer between a positive electrode and a light-emitting layer, and a device including an electron transport layer or a hole block layer between a negative electrode and a light-emitting layer, and including a hole transport layer or an electron block layer between a positive electrode and the light-emitting layer.

Specific structures of the device of the present invention are shown below. Here, the symbol “/” represents the fact that respective layers on both side of the “/” are stacked adjacent to each other. Hereinafter, the same applies.

a) positive electrode/(charge injection layer)/light-emitting layer/(charge injection layer)/negative electrode
b) positive electrode/(charge injection layer)/hole transport layer/light-emitting layer/(charge injection layer)/negative electrode
c) positive electrode/(charge injection layer)/light-emitting layer/electron transport layer/(charge injection layer)/negative electrode
d) positive electrode/(charge injection layer)/hole transport layer/light-emitting layer/electron transport layer/(charge injection layer)/negative electrode

Further, in the device of the present invention, two or more layers of the light-emitting layer, hole transport layer, and electron transport layer may be each independently provided.

Of the charge transport layers (hole transport layer and electron transport layer) arranged adjacent to an electrode, a charge transport layer having a function for improving a charge injection efficiency from the electrode and an effect for reducing a driving voltage of the device may be generally referred to as a charge injection layer (hole injection layer and electron injection layer). Examples of devices having a charge injection layer may include a device including a charge injection layer adjacent to the negative electrode and a device including a charge injection layer adjacent to the positive electrode.

In the device of the present invention, an insulation layer having a thickness of 2 nm or less may be provided adjacent to an electrode in order to improve adhesion with the electrode or to improve charge injection from the electrode. Examples of the material used for the insulation layer may include a metal fluoride, a metal oxide, and an organic insulating material. Examples of the devices having an insulation layer with a thickness of 2 nm or less may include a device including an insulation layer adjacent to the negative electrode and a device including an insulation layer adjacent to the positive electrode.

To improve interfacial adhesion and to prevent layer mixing, the device of the present invention may be further provided with a buffer layer having an average film thickness of 2 nm or less between an electrode and the light-emitting layer adjacent to the electrode, or on the interface between the electron transport later and the light-emitting layer.

The respective layers in the device of the present invention will now be described.

### (Light-Emitting Layer)

The above-described light-emitting layer may be a layer which uses the metal complex of the present invention, or the composition of the present invention. In other words, it may be the organic thin film of the present invention. The light-emitting layer may be formed from a single layer or from a plurality of layers. Also, the light-emitting layer may be formed from only the metal complex or composition of the present invention, or may be formed from a mixture that comprises another light-emitting material in addition to the metal complex or composition of the present invention. The light-emitting layer may further include at least one layer comprising the metal complex or composition of the present invention. Examples of the other light-emitting material that may be comprised in the light-emitting layer may include naphthalene derivatives, anthracene and derivatives thereof, perylene and derivatives thereof, pigments such as polymethine, xanthene, coumarin, and cyanine pigments, 8-hydroxyquinoline and metal complexes of derivatives thereof, aromatic amines, tetraphenylcyclopentadiene and derivatives thereof, and tetraphenylbutadiene and derivatives thereof.

### (Hole Transport Layer)

Examples of the material used for the hole transport layer may include the compounds described in Japanese Patent Application Laid-Open Nos. Sho. 63-70257, Sho. 63-175860, Hei. 2-135359, Hei. 2-135361, Hei. 2-209988, Hei. 3-37992, and Hei. 3-152184. Specifically, examples of this material include polyvinyl carbazole and derivatives thereof, polysilane and derivatives thereof, polysiloxane derivatives having an aromatic amine compound group on a side chain or a main chain, pyrazoline derivatives, arylamine derivatives, stilbene derivatives, triphenyldiamine derivatives, polyaniline and derivatives thereof, polyaminophen and derivatives thereof, polypyrrole and derivatives thereof, poly(p-phenylenevinylene) and derivatives thereof, and poly(2,5-thienylenevinylene) and derivatives thereof.

The thickness of the hole transport layer is appropriately set so that a light-emitting efficiency or photoelectric efficiency and a driving voltage are suitable values. Although the optimum value depends on the used materials, a thickness at which pin holes do not form is necessary. If the thickness of the hole transport layer is too thick, the driving voltage of the device tends to increase. Therefore, the thickness of the hole transport layer is preferably 1 nm to 1 μm, more preferably 2 to 500 nm, and particularly preferably 5 to 200 nm.

### (Electron Transport Layer)

Examples of the material used for the electron transport layer may include the compounds described in Japanese Patent Application Laid-Open Nos. Sho. 63-70257, Sho. 63-175860, Hei. 2-135359, Hei. 2-135361, Hei. 2-209988, Hei. 3-37992, and Hei. 3-152184. Specifically, examples of this material include oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and metal complexes of derivatives thereof, polyquinoline and derivatives thereof, polyquinoxaline and derivatives thereof, and polyflorene and derivatives thereof.

The thickness of the electron transport layer is appropriately set so that a light-emitting efficiency or photoelectric efficiency and a driving voltage are suitable values. Although the optimum value depends on the used materials, a thickness at which pin holes do not form is necessary. If the thickness of the electron transport layer is too thick, the driving voltage of the device tends to increase. Therefore, the thickness of the electron transport layer is preferably 1 nm to 1 μm, more preferably 2 to 500 nm, and particularly preferably 5 to 200 nm.

### (Substrate)

The device of the present invention is usually formed using a substrate. An electrode is formed on one surface of the substrate, and the respective layers of the device are formed on the other surface of the substrate. The substrate used in the present invention is a substrate that does not chemically change during formation of the electrodes and the respective layers. Examples of this substrate may include substrates formed from glass, plastic, polymer film, and silicon. If the substrate is non-transparent, it is preferable to form a transparent or translucent electrode as the opposite electrode.

### (Electrodes)

Generally, It is preferable that at least one of the positive electrode and the negative electrode is transparent or translucent, and that the positive electrode is transparent or translucent. Further, if the device of the present invention is a photovoltaic device, at least one of the negative electrode and the positive electrode may be formed in a comb shape. In this case, although the electrodes may be non-transparent, they are preferably transparent or translucent.

Examples of the material used for the positive electrode may include a conductive metal oxide film and a translucent metal thin film. Specifically, examples of this material include indium oxide, zinc oxide, tin oxide and composites thereof (indium tin oxide (ITO), indium zinc oxide etc.), antimony tin oxide, NESA, gold, platinum, silver, and copper. Among these, ITO, indium zinc oxide, and tin oxide are preferable. Further, an organic transparent conductive film may be used for the positive electrode, including polyaniline and derivatives thereof, and polyaminophen and derivatives thereof.

Examples of the method of forming the positive electrode may include a vacuum deposition method, a sputtering method, an ion plating method, and a plating method.

The thickness of the positive electrode can be appropriately set in consideration of light permeability and electrical conductivity. For example, it is preferably 10 nm to 10 μm, more preferably 20 nm to 1 μm, and particularly preferably 50 to 500 nm.

The material used for the negative electrode preferably has a small work function. Examples of this material may include metals such as lithium, sodium, potassium, rubidium, cesium, beryllium, magnesium, calcium, strontium, barium, aluminum, scandium, vanadium, zinc, yttrium, indium, cerium, samarium, europium, terbium, and ytterbium; an alloy of two or more of these metals; an alloy of one or more of these metals and one or more of gold, silver, platinum, copper, manganese, titanium, cobalt, nickel, tungsten, and tin; graphite; and intercalated graphite compounds. Examples of the above-described alloys may include a magnesium-silver alloy, a magnesium-indium alloy, a magnesium-aluminum alloy, an indium-silver alloy, a lithium-aluminum alloy, a lithium-magnesium alloy, a lithium-indium alloy, and a calcium-aluminum alloy.

Examples of the method for forming the positive electrode and the negative electrode may include a vacuum deposition method, a sputtering method, and a method of laminating by thermal compression bonding of a metal thin film. Further, a negative electrode having a layered structure of two or more layers may also be formed.

The thickness of the negative electrode can be appropriately set in consideration of electrical conductivity and durability. It is preferably 10 nm to 10 μm, more preferably 20 nm to 1 μm, and particularly preferably 50 to 500 nm.

Further, a layer which is formed from conductive polymers, or a layer having an average thickness of 2 nm or less which is formed from a metal oxide, a metal fluoride, an organic insulating material or the like may be provided between the negative electrode and the organic material layer.

### (Protective Layer)

In the device of the present invention, a protective layer and/or protective cover that protects the device may be formed after forming the negative electrode, in o externally protect the device of the present invention so as to allow long term use.

Examples of the material used for such a protective layer may include a macromolecular compound, a metal oxide, a metal fluoride, and a metal boride. Examples of the protective cover may include a glass plate, and a plastic sheet whose surface is subjected to a treatment for low water permeability. Among these, it is preferred to seal the device by laminating a protective cover and the device by using a thermosetting resin or a photocurable resin.

### (Charge Injection Layer)

Examples of the charge injection layer may include a layer including a conductive polymer, a layer containing a material that has an ionization potential with a value between that of the material for the positive electrode and the material for the hole transport which is comprised in the hole transport layer (when it is provided between the positive electrode and the hole transport layer), and a layer containing a material that has an electron affinity with a value between that of the material for the negative electrode and the material for the electron transport which is included in the electron transport layer (when it is provided between the negative electrode and the electron transport layer).

The material used in the electron injection layer may be selected based on the relationship with the materials in the electrodes and adjacent layers. Specifically, examples of this material may include polyaniline and derivatives thereof, polyaminophen and derivatives thereof, polypyrrole and derivatives thereof, polyphenylenevinylene and derivative thereof, polythienylenevinylene and derivatives thereof, polyquinoline and derivatives thereof, polyquinoxaline and derivatives thereof, conductive polymers such as polymers having an aromatic amine structure on a main chain or a side chain, metal phthalocyanines (copper phthalocyanine etc.), and carbon.

The thickness of the charge injection layer is preferably 1 nm to 100 nm, and more preferably 2 nm to 50 nm.

If the device of the present invention is a light-emitting device, such a light-emitting device may be used as a surface light source, a backlight for a segment display apparatus, a dot-matrix display apparatus or a liquid crystal display apparatus, or an lluminator.

To obtain a surface emission using this light-emitting device, a planar positive electrode and negative electrode can be arranged superimposed over each other. Further, examples of methods that may be used to obtain a patterned emission include a method of mounting a mask provided with a patterned window on the surface of a surface light-emitting device, a method of forming a portion that essentially does not emit light by forming part of the organic layer to be much thicker, and a method of forming one or both of the positive electrode and the negative electrode in a pattern. By forming a pattern with any of these methods and arranging some of the electrodes so that they can be independently switched on/off, a segment display device can be obtained that is capable of displaying numbers, characters, and simple symbols. Further, a dot-matrix display device can be obtained by orthogonally arranging the positive electrode and the negative electrode in a stripe-shape pattern.

In the dot-matrix display device, a partial color display or a multi-color display can be achieved by coating light-emitting materials in a plurality of different emission colors or by using a color filter or a light conversion filter. Further, the dot-matrix display device can be passively driven, or even actively driven by combining with a TFT and the like. These display devices can be used in a display apparatus for a computer, a television, a handheld terminal, a cellular phone, a car navigation system, a video camera view finder and the like.

The surface light-emitting device is a self light-emitting thin-type device, which can be preferably used as a surface light source for a backlight of a liquid crystal display apparatus, or a planar illumination light source. Further, by using a flexible substrate, this light-emitting device can also be used as a curved light source or display apparatus.

If the device of the present invention is a switching device, this switching device can be used in a liquid crystal display apparatus having an active matrix drive circuit.

If the device of the present invention is a photovoltaic device, this photovoltaic device can be used in a solar cell.

Since the metal complex of the present invention is useful as a magnetic material, the metal complex is also useful as a biological probe and a contrast agent. Further, the metal complex of the present invention is useful as a material such as an additive, a modifier, and a catalyst.

### EXAMPLES

The present invention will now be described in more detail with the following examples.

The ultraviolet-visible absorption spectrum was determined by measuring with an absorption spectrophotometer (Cary 5E manufactured by Varian). The emission spectrum was measured with a spectrophotofluorometer (trade name: FP-6500 manufactured by Jasco Corporation) at an excitation wavelength of 389 nm. The emission quantum yield was calculated by comparing with the emission quantum yield (55%) for 1N aqueous sulfuric acid solution of quinine sulfate as a standard sample. The excitation life was determined as the excitation life at an emission peak wavelength of the emission spectrum as obtained from a spectrophotofluorometer (trade name: Fluorolog-Tau3 manufactured by JOBINYVON-SPEX).

### Synthesis Example 1

The above-described ligand represented by formula (B-1) was synthesized according to the description in the Journal of American Chemical Society 106, 4765 to 4772 (1984). A mixture of 1,2-diaminobenzene and 2-hydroxy-1,3-diaminopropane tetraacetic acid was reacted by heating at 170 to 180° C. for 1 hour. Then, the resultant product and ethyl bromide were left for 2 days in tetrahydrofuran solution in the presence of sodium hydroxide, to obtain the above-described ligand represented by formula (B-1).

### Synthesis Example 2

The above-described ligand represented by formula (B-2) was synthesized according to the description in the Journal of American Chemical Society 109, 5227 to 5233 (1987). A mixture of 1,2-diaminobenzene and 2-hydroxy-1,3-diaminopropane tetraacetic acid was reacted by heating at 170 to 180° C. for 1 hour to obtain the above-described ligand represented by formula (B-2).

### Synthesis Example 3

The above-described ligand represented by formula (B-9) was synthesized according to the description in the Journal of American Chemical Society 104, 3607 to 3617 (1982) and in Tetrahedron Letter, 29, 3033 to 3036. A mixture of 1,2-diaminobenzene, ethylenediaminetetraacetic acid, and ethylene glycol was reacted by heating at 200° C. for 22 hours. Then, the resultant product and 1-bromopropane were reacted for 3 hours in dimethyl sulfoxide solution at room temperature in the presence of potassium hydroxide, to obtain the above-described ligand represented by formula (B-9).

### Synthesis Example 4

The above-described ligand represented by formula (B-10) was synthesized according to the description in the Journal of American Chemical Society 104, 3607 to 3617 (1982) and in Tetrahedron Letter, 29, 3033 to 3036. A mixture of 1,2-diaminobenzene, 1,3-propane-N,N,N′,N′-tetraacetic acid, and ethylene glycol was reacted by heating at 200° C. for 22 hours. Then, the resultant product and 1-bromopropane were reacted for 3 hours in dimethyl sulfoxide solution at room temperature in the presence of potassium hydroxide, to obtain the above-described ligand represented by formula (B-10).

### Synthesis Example 5

The above-described ligand represented by formula (B-11) was synthesized according to the description in the Journal of American Chemical Society, Dalton Transaction, 2579 to 2593 (1987) and in Tetrahedron Letter, 29, 3033 to 3036. A mixture of 1,2-diaminobenzene and ethylene glycol bis(2-aminoethylether)-N,N,N′,N′-tetraacetic acid was reacted by heating at 180° C. for 4 hours. Then, the resultant product and 1-bromopropane were reacted for 1.5 hours in dimethyl sulfoxide solution at room temperature in the presence of potassium hydroxide, to obtain the above-described ligand represented by formula (B-11).

### Synthesis Example 6

The above-described ligand represented by formula (B-12) was synthesized according to the description in the Journal of American Chemical Society, Dalton Transaction, 2579 to 2593 (1987). A mixture of 1,2-diaminobenzene and trans-1,2-cyclohexanediamine-N,N,N′,N′-tetraacetic acid was reacted by heating at 180° C. for 3 hours, to obtain the above-described ligand represented by formula (B-12).

### Synthesis Example 7

The above-described ligand represented by formula (B-13) was synthesized according to the description in the Journal of American Chemical Society, Dalton Transaction, 2579 to 2593 (1987) and in Tetrahedron Letter, 29, 3033 to 3036. A mixture of 1,2-diaminobenzene and trans-1,2-cyclohexanediamine-N,N,N′,N′-tetraacetic acid was reacted by heating at 180° C. for 3 hours. Then, the resultant product and 1-bromopropane were reacted for 3 hours in dimethyl sulfoxide solution at room temperature in the presence of potassium hydroxide, to obtain the above-described ligand represented by formula (B-13).

### Example 1

The above-described ligand represented by formula (B-1) (500 mg, 0.692 mmol) and cerium trifluoromethanesulfonate (406 mg, 0.692 mmol) were charged into a flask. Then, 1 mL of acetonitrile was added thereto and dissolved. The mixed solution was stirred for 30 minutes at room temperature, and the solvent was then evaporated in vacuo. The residue was dissolved in 10 mL of dichloromethane. Thereto, 15 mL of diethyl ether was added while vigorously shirring, to obtain a precipitate. The obtained precipitate was collected and dried in vacuo to obtain the above-described metal complex represented by composition formula (C-1) (hereinafter, “metal complex (C-1)”). The collected amount was 501 mg (yield 55%).

Elemental analysis: Found (%) C, 40.75; H, 4.04; N, 10.34; S, 7.63; F, 12.92; Ce, 9.62. Calcd for C₄₆H₅₆CeF₉N₁₀O₁₃S₃ (%) C, 40.50; H, 4.14; N, 10.27; S, 7.05; F, 12.53; Ce, 10.27.

The metal complex (C-1) emitted an aqua blue color in a solid powder state and in a solution state (acetonitrile, ethanol, and methanol) under ultraviolet excitation (365 nm).

The emission spectrum in acetonitrile had a peak at 434 nm, the emission quantum yield was 17%, and the excitation life was 33.0 ns.

### Example 2

The above-described ligand represented by formula (B-2) (500 mg, 0.819 mmol) and cerium trifluoromethanesulfonate (481 mg, 0.819 mmol) were charged into a flask. Then, acetonitrile (1 mL) was added thereto and dissolved. The mixed solution was stirred for 30 minutes at room temperature. Then, while vigorously shirring, 15 mL of dichloromethane was added to form a precipitate. The precipitate was collected and dried in vacuo to obtain the above-described metal complex represented by composition formula (C-2) (hereinafter, “metal complex (C-2)”). The collected amount was 750 mg (yield 76%).

Elemental analysis: Found (%) C, 37.70; H, 3.19; N, 11.62; S, 7.59; F, 13.00; Ce, 10.8. Calcd for C₃₈H₃₆CeF₉N₁₀O₁₁S₃ (%) C, 37.53; H, 2.98; N, 11.52; S, 7.91; F, 14.06; Ce, 11.52.

The metal complex (C-2) emitted an aqua blue color in a solid powder state and in a solution state (acetonitrile, ethanol, and methanol) under ultraviolet excitation (365 nm).

The emission spectrum in acetonitrile had a peak at 433 nm, the emission quantum yield was 25%, and the excitation life was 31.0 ns.

### Example 3

The above-described ligand represented by formula (B-9) (50 mg, 0.069 mmol) and cerium trifluoromethanesulfonate (46 mg, 0.078 mmol) were charged into a flask. Then, ethanol (4 mL) was added thereto and dissolved. The mixed solution was stirred for 2.5 hours at room temperature, and then the stirring was stopped. About 4 mL of diethyl ether was added, and the resultant mixture was left to stand overnight. After that, the produced solid was collected to obtain the above-described metal complex represented by composition formula (C-9) (hereinafter, “metal complex (C-9)”). The collected amount was 63 mg (yield 71%).

Elemental analysis: Found (%) C, 43.59; H, 4.36; N, 10.47; S, 7.49. Calcd for C₄₉H₅₈CeF₉N₁₀O₁₀S₃ (%) C, 43.45; H, 4.32; N, 10.34; S, 7.10.

The metal complex (C-9) emitted a blue color in a solid powder state and in a solution state (acetonitrile) under ultraviolet excitation (365 nm).

The emission spectrum in acetonitrile had a peak at 421.5 nm, the emission quantum yield was 9.8%, and the excitation life was 68.2 ns.

### Example 4

The above-described ligand represented by formula (B-10) (50 mg, 0.066 mmol) and cerium trifluoromethanesulfonate (35 mg, 0.060 mmol) were charged into a flask. Then, ethanol (4 mL) was added thereto and dissolved. The mixed solution was stirred for 2.5 hours at room temperature, and then the stirring was stopped. About 4 mL of diethyl ether was added, and the resultant mixture was left to stand overnight. After that, the produced solid was collected to obtain the above-described metal complex represented by composition formula (C-10) (hereinafter, “metal complex (C-10)”). The collected amount was 51 mg (yield 63%).

Elemental analysis: Found (%) C, 44.27; H, 4.39; N, 10.30; S, 7.33. Calcd for C₅₀H₅₈CeF₉N₁₀O₉S₃ (%) C, 44.47; H, 4.33; N, 10.37; S, 7.12.

The metal complex (C-10) emitted a blue color in a solid powder state and in a solution state (acetonitrile) under ultraviolet excitation (365 nm).

The emission spectrum in acetonitrile had a peak at 421 nm, the emission quantum yield was 21%, and the excitation life was 73.8 ns.

### Example 5

The above-described ligand represented by formula (B-11) (28 mg, 0.033 mmol) and cerium trifluoromethanesulfonate (18 mg, 0.030 mmol) were charged into a flask. Then, ethanol (2 mL) was added thereto and dissolved. The mixed solution was stirred for 2.5 hours at room temperature, and then the stirring was stopped. About 4 mL of diethyl ether was added, and the resultant mixture was left to stand overnight. After that, the produced solid was collected to obtain the above-described metal complex represented by composition formula (C-11) (hereinafter, “metal complex (C-11)”). The collected amount was 27 mg (yield 63%).

Elemental analysis: Found (%) C, 43.12; H, 4.84; N, 9.15; S, 6.30. Calcd for C₅₃H₆₄CeF₉N₁₀O₁₁S₃ (%) C, 43.33; H, 5.02; N, 9.19; S, 6.31.

The metal complex (C-11) emitted a blue color in a solid powder state and in a solution state (acetonitrile) under ultraviolet excitation (365 nm).

The emission spectrum in acetonitrile had a peak at 406.5 nm, the emission quantum yield was 1.8%, and the excitation life was 62.7 ns.

### Example 6

The above-described ligand represented by formula (B-12) (50 mg, 0.079 mmol) and cerium trifluoromethanesulfonate (45 mg, 0.077 mmol) were charged into a flask. Then, acetonitrile (5 mL) was added thereto and dissolved. The mixed solution was stirred for 2 hours at room temperature, and then the stirring was stopped. About 150 mL of diethyl ether was added, and the resultant mixture was left to stand overnight. About 10 mL of hexane was further added, and the solid produced by leaving the mixture to stand overnight was recovered to obtain the above-described metal complex represented by composition formula (C-12) (hereinafter, “metal complex (C-12)”). The collected amount was 14 mg (yield 15%).

Elemental analysis: Found (%) C, 39.96; H, 3.23; N, 11.35; S, 8.35. Calcd for C₄₁H₃₈CeF₉N₁₀O₉S₃ (%) C, 40.29; H, 3.13; N, 11.46; S, 7.87.

The metal complex (C-12) emitted a blue color in a solid powder state and in a solution state (acetonitrile) under ultraviolet excitation (365 nm).

The emission spectrum in acetonitrile had a peak at 428 nm, the emission quantum yield was 24%, and the excitation life was 42 ns.

### Example 7

The above-described ligand represented by formula (B-13) (50 mg, 0.062 mmol) and cerium trifluoromethanesulfonate (35 mg, 0.060 mmol) were charged into a flask. Then, ethanol (5 mL) was added thereto and dissolved. The mixed solution was stirred for 2 hours at room temperature, and then the stirring was stopped. About 20 mL of diethyl ether was added, and the resultant mixture was left to stand overnight. After that, the produced solid was collected to obtain the above-described metal complex represented by composition formula (C-13) (hereinafter, “metal complex (C-13)”). The collected amount was 41 mg (yield 43%).

Elemental analysis: Found (%) C, 45.67; H, 4.71; N, 9.93; S, 6.54. Calcd for C₅₃H₆₂CeF₉N₁₀O₉S₃ (%) C, 45.78; H, 4.49; N, 10.07; S, 6.92.

The metal complex (C-13) emitted a blue color in a solid powder state and in a solution state (acetonitrile) under ultraviolet excitation (365 nm).

The emission spectrum in acetonitrile had a peak at 428.5 nm, the emission quantum yield was 25%, and the excitation life was 56 ns.

### <Emission Spectrum>

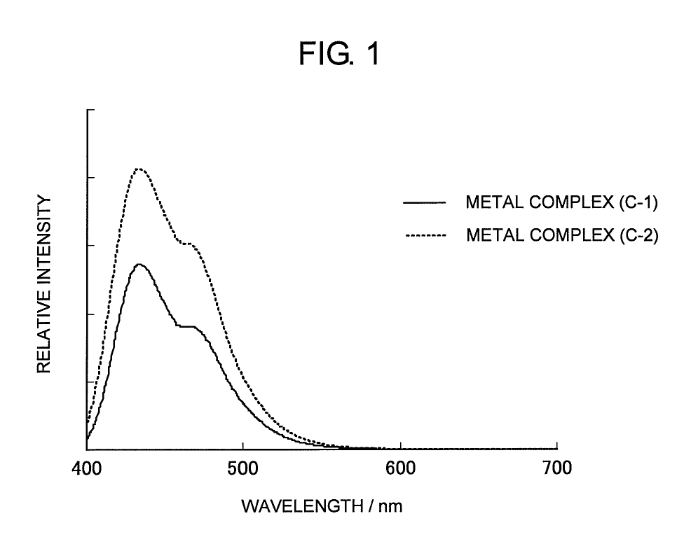

FIG. 1 illustrates the emission spectra in acetonitrile of the metal complex (C-1) and the metal complex (C-2).

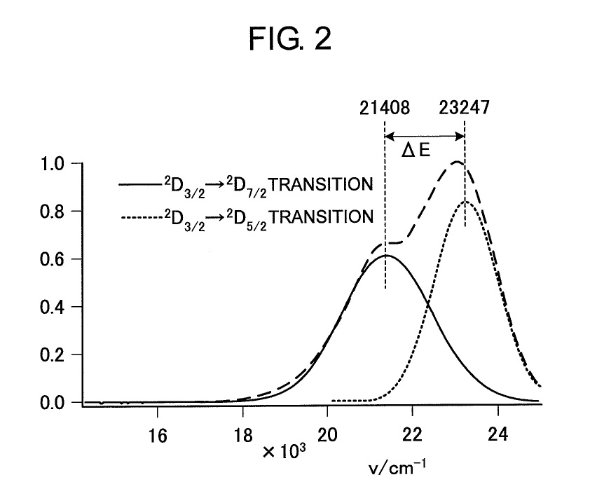

FIG. 2 illustrates the fitting results of the spectrum of the metal complex (C-2) based on two Gaussian functions. The peak interval between these Gaussian functions is 1840 cm⁻¹, indicating a difference in energy states of ²F_{7/2} and ²F_{5/2} of a cerium ion. In other words, it is shown that this emission was derived from the formed complex.

### <Temperature Durability>

The metal complex (D-1) represented below was synthesized according to the description in Angew. Chem. Int. Ed. 46, 7399 to 7403 (2007). In the emission spectra of the metal complex (D-1) and the metal complexes (C-1), (C-9), (C-10), (C-11) and (C-13) in acetonitrile solution (both concentrations are 6 μM), when the temperature was increased from 35° C. to 50° C., the emission intensity for the metal complexes (C-1), (C-9), (C-10), (C-11) and (C-13) only decreased by 1% or less, although the emission intensity for the metal complex (D-1) decreased by about 6%.

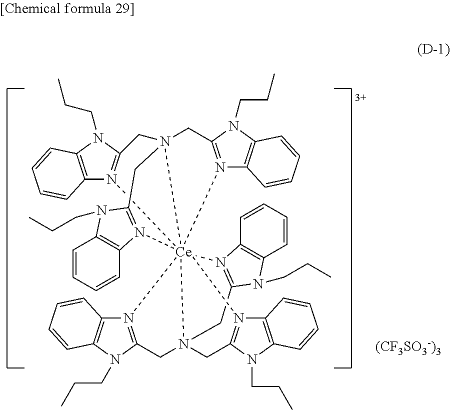

### <Solubility>

The solubilities of the metal complexes (C-1), (C-9), (C-10), (C-11), (C-12) and (C-13), and the solubility of the metal complex (D-1) were tested in an organic solvent. Specifically, the metal complexes (C-1), (C-9), (C-10), (C-11), (C-12) and (C-13), and the metal complex (D-1) were examined to see whether they are dissolved in chloroform at 25° C. The results showed that the metal complexes (C-1), (C-9), (C-10), (C-11), (C-12), and (C-13) were readily soluble in chloroform, although the metal complex (D-1) was hardly soluble in chloroform.

### INDUSTRIAL APPLICABILITY

The metal complex of the present invention is useful as a material for a light-emitting device, a switching device, a photovoltaic device, a biological probe, a contrast agent, an additive, a modifier, a catalyst and the like.

1. A metal complex comprising:
(a) a polydentate ligand having denticity of five or more that includes a heteroaromatic ring which contains two or more atoms selected from the group consisting of a nitrogen atom, an oxygen atom and a sulfur atom; and
(b) an ion of a metal selected from the group consisting of cerium, praseodymium, ytterbium, and lutetium.

2. The metal complex according to claim 1, wherein the number of said polydentate ligand comprised in said metal complex is one.

3. The metal complex according to claim 1, wherein said heteroaromatic ring is an imidazole ring or a condensed imidazole ring.

4. The metal complex according to claim 1, wherein said polydentate ligand is represented by the following formula (1): 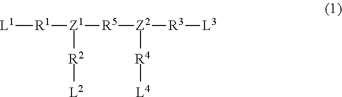
wherein
R¹, R², R³, R⁴, and R⁵ each independently represent a divalent group or a direct bond;
Z¹ and Z² each independently represent a nitrogen atom, a phosphorus atom, or a trivalent group; and
L¹, L², L³, and L⁴ each independently represent a coordinating group or a hydrogen atom;
wherein at least one of L¹, L², L³, and L⁴ is a coordinating group represented by the following formula (2): 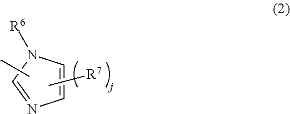
wherein
R⁶ represents a hydrogen atom or a substituent;
R⁷ represents a substituent; and
j represents an integer of from 0 to 2; and
when R⁶ and R⁷ each represent a substituent bonded to atoms adjacent to each other, R⁶ and R⁷ may be linked to form a ring; and
when j is 2 and two R⁷s each represent a substituent bonded to carbon atoms adjacent to each other, two R⁷s may be linked together to form a ring;
or at least one of L¹, L², L³, and L⁴ is a coordinating group represented by the following formula (3): 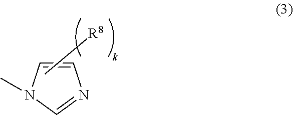
wherein
R⁸ represents a substituent; and
k is an integer of from 0 to 3; and
when k is 2 and R⁸s each represent a substituent bonded to carbon atoms adjacent to each other, R⁸s may be linked to form a ring; and
when k is 3, R⁸ bonded to the carbon atom at position 4 and R⁸ bonded to the carbon atom at position 5 may be linked together to form a ring.

5. The metal complex according to claim 1, wherein said metal complex is represented by the following composition formula (4): 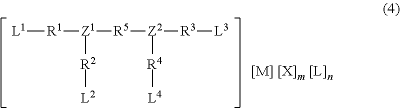
wherein
M represents an ion of a metal selected from the group consisting of cerium, praseodymium, ytterbium, and lutetium,
X represents a counter ion;
L represents a ligand having denticity of 4 or less; and
m is an integer of from 0 to 4, and n is an integer of 0 or more.

6. The metal complex according to claim 4, wherein R¹, R², R³, R⁴, and R⁵ in said polydentate ligand each independently represent a divalent group represented by the following formula (5): 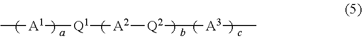
wherein
Q¹ and Q² each independently represent a divalent hydrocarbyl group that is optionally substituted, or a divalent heterocyclyl group that is optionally substituted; and
A¹, A², and A³ each independently represent a group represented by the following formula: 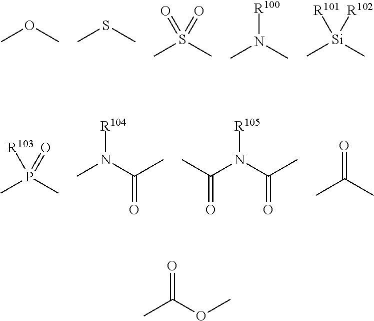
wherein
R¹⁰⁰, R¹⁰⁴, and R¹⁰⁵ each represent a hydrocarbyl group that is optionally substituted;
R¹⁰¹ and R¹⁰² each independently represent a hydrocarbyl group that is optionally substituted, or a hydrocarbyloxy group that is optionally substituted;
R¹⁰³ represents a hydrocarbyl group that is optionally substituted, or a hydrocarbyloxy group that is optionally substituted; and
a and c are each independently 0 or 1, and b is an integer of from 0 to 10.

7. The metal complex according to claim 4, wherein R¹, R², R³, and R⁴ in said polydentate ligand each independently represent a divalent hydrocarbyl group that is optionally substituted.

8. The metal complex according to claim 4, wherein Z¹ and Z² in said polydentate ligand are each a nitrogen atom.

9. The metal complex according to claim 4, wherein said polydentate ligand is represented by the following formula (6): 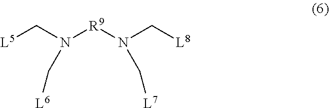
wherein
R⁹ represents a divalent group; and
L⁵, L⁶, L⁷, and L⁸ each independently represent a coordinating group or a hydrogen atom;
wherein at least one of L⁵, L⁶, L⁷, and L⁸ is said coordinating group represented by formula (2) or (3).

10. The metal complex according to claim 4, wherein L¹, L², L³, and L⁴ in said polydentate ligand are each independently said coordinating group represented by formula (2) or (3).

11. The metal complex according to claim 4, wherein said polydentate ligand is represented by the following formula (7): 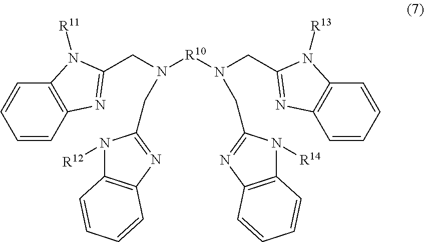
wherein
R¹⁰ represents a divalent group; and
R¹¹, R¹², R¹³, and R¹⁴ each independently represent a hydrogen atom or a substituent.

12. The metal complex according to claim 1, wherein said metal complex is represented by the following composition formula (8): 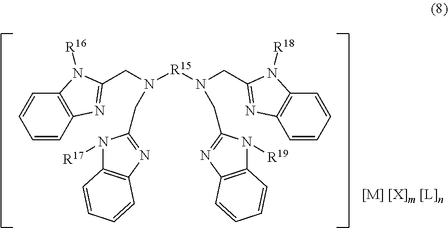
wherein
R¹⁵ represents a divalent group;
R¹⁶, R¹⁷, R¹⁸ and R¹⁹ each independently represent a hydrogen atom or a substituent;
M represents an ion of a metal selected from the group consisting of cerium, praseodymium, ytterbium, and lutetium;
X represents a counter ion;
L represents a ligand having denticity of 4 or less; and
m is an integer of from 0 to 4, and n is an integer of 0 or more.

13. The metal complex according to claim 1, wherein said metal is cerium.

14. A composition comprising the metal complex according to claim 1 and a charge transport material.

15. An organic thin film obtained by using the metal complex according to claim 1.

16. A device obtained by using the metal complex according to claim 1.
